(19) United States
(12) Patent Application Publication
(10) Pub. No.: US 2013/0144346 A1
(43) Pub. Date: Jun. 6, 2013

(54) Modular polyaxial bone anchor with retainer having interconnected pieces

(71) Applicants: Roger P. Jackson, Prairie Village, KS (US); James L. Surber, Kansas City, KS (US)

(72) Inventors: Roger P. Jackson, Prairie Village, KS (US); James L. Surber, Kansas City, KS (US)

(21) Appl. No.: 13/694,953

(22) Filed: Jan. 22, 2013

Related U.S. Application Data
(63) Continuation-in-part of application No. 13/694,032, filed on Oct. 22, 2012.
Application No. 13/694,032 is a continuation of application No. 12/804,999, filed on Aug. 3, 2010, now Pat. No. 8,308,782.
Application No. 12/804,999 is a continuation-in-part of application No. 12/080,202, filed on Apr. 1, 2008, now Pat. No. 7,875,065.
Application No. 12/080,202 is a continuation-in-part of application No. 11/281,818, filed on Nov. 17, 2005, now Pat. No. 7,625,396.
Application No. 12/804,999 is a continuation-in-part of application No. 12/229,207, filed on Aug. 20, 2008, now Pat. No. 8,353,932.
Application No. 12/229,207 is a continuation-in-part of application No. 11/522,503, filed on Sep. 14, 2006, now Pat. No. 7,766,915.
Application No. 11/522,503 is a continuation-in-part of application No. 11/024,543, filed on Dec. 20, 2004, now Pat. No. 7,204,838.
(60) Provisional application No. 61/633,385, filed on Feb. 9, 2012.
(60) Provisional application No. 61/273,399, filed on Aug. 4, 2009.
(60) Provisional application No. 60/630,478, filed on Nov. 23, 2004.
(60) Provisional application No. 60/994,083, filed on Sep. 17, 2007.

(51) Int. Cl. A61B 17/86

(52) U.S. Cl. CPC A61B 17/8605
USPC 606/305

(57) ABSTRACT

A polyaxial bone screw assembly includes a threaded shank body having an integral upper portion receivable in a one-piece receiver, the receiver having an upper channel for receiving a longitudinal connecting member and a lower cavity cooperating with a lower opening. A down-loadable, friction fit compression insert (some with lock and release feature), a down-loadable two-piece, interconnected retaining ring articulatable with respect to the receiver and an up-loadable shank upper portion cooperate to provide for assembly of the shank with the receiver either prior to or after implantation of the shank into a vertebra.

### CROSS-REFERENCE TO RELATED APPLICATIONS

This application claims the benefit of U.S. Provisional Patent Application Ser. No. 61/633,385 filed Feb. 9, 2012, the disclosure of which is incorporated by reference herein.

This application is also a continuation-in-part of U.S. patent application Ser. No. 13/694,032 filed Oct. 22, 2012 that is a continuation of U.S. patent application Ser. No. 12/804,999, filed Aug. 3, 2010, now U.S. Pat. No. 8,308,782, that claims the benefit of U.S. Provisional Patent Application Ser. No. 61/273,399, filed Aug. 4, 2009, all of the disclosures of which are incorporated by reference herein. U.S. patent application Ser. No. 12/804,999 is also a continuation-in-part of U.S. patent application Ser. No. 12/080,202 filed Apr. 1, 2008, now U.S. Pat. No. 7,875,065, that is a continuation-in-part of U.S. patent application Ser. No. 11/281,818 filed Nov. 17, 2005, now U.S. Pat. No. 7,625,396, that claims the benefit of U.S. Provisional Patent Application Ser. No. 60/630,478 filed Nov. 23, 2004, all of the disclosures of which are incorporated by reference herein. U.S. patent application Ser. No. 12/804,999 is also a continuation-in-part of U.S. patent application Ser. No. 12/229,207 filed Aug. 20, 2008 that claims the benefit of U.S. Provisional Patent Application Ser. No. 60/994,083 filed Sep. 17, 2007, all of the disclosures of which are incorporated by reference herein. U.S. patent application Ser. No. 12/804,999 is also is a continuation-in-part of U.S. patent application Ser. No. 11/522,503 filed Sep. 14, 2006, now U.S. Pat. No. 7,766,915, that is a continuation-in-part of U.S. patent application Ser. No. 11/024,543 filed Dec. 20, 2004, now U.S. Pat. No. 7,204,838, all of the disclosures of which are incorporated by reference herein.

### BACKGROUND OF THE INVENTION

The present invention is directed to polyaxial bone anchors for use in bone surgery, particularly spinal surgery and particularly to such bone anchors with compression or pressure inserts and further including retainers for capturing and retaining a bone screw shank head in the receiver member assembly and later fixing the bone screw shank with respect to the receiver assembly.

Bone screws are utilized in many types of spinal surgery in order to secure various implants to vertebrae along the spinal column for the purpose of stabilizing and/or adjusting spinal alignment. Although both closed-ended and open-ended bone screws are known, open-ended screws are particularly well suited for connections to rods and connector arms, because such rods or arms do not need to be passed through a closed bore, but rather can be laid or urged into an open channel within a receiver or head of such a screw. Generally, the screws must be inserted into the bone as an integral unit along with the head, or as a preassembled unit in the form of a shank and pivotal receiver, such as a polyaxial bone screw assembly.

Typical open-ended bone screws include a threaded shank with a pair of parallel projecting branches or arms which form a yoke with a U-shaped slot or channel to receive a rod. Hooks and other types of connectors, as are used in spinal fixation techniques, may also include similar open ends for receiving rods or portions of other fixation and stabilization structure.

A common approach for providing vertebral column support is to implant bone screws into certain bones which then in turn support a longitudinal structure such as a rod, or are supported by such a rod. Bone screws of this type may have a fixed head or receiver relative to a shank thereof, or may be of a polyaxial screw nature. In the fixed bone screws, the rod receiver head cannot be moved relative to the shank and the rod must be favorably positioned in order for it to be placed within the receiver head. This is sometimes very difficult or impossible to do. Therefore, polyaxial bone screws are commonly preferred. Open-ended polyaxial bone screws typically allow for a loose or floppy rotation of the head or receiver about the shank until a desired rotational position of the receiver is achieved by fixing such position relative to the shank during a final stage of a medical procedure when a rod or other longitudinal connecting member is inserted into the receiver, followed by a locking screw or other closure. This floppy feature can be, in some cases, undesirable and make the procedure more difficult. Also, it is often desirable to insert the bone screw shank separate from the receiver or head due to its bulk which can get in the way of what the surgeon needs to do. Such screws that allow for this capability are sometimes referred to as modular polyaxial screws.

With specific reference to modular snap-on or pop-on polyaxial pedicle screw systems having shank receiver assemblies, the prior art has shown and taught the concept of the receiver and certain retainer parts forming an assembly wherein a contractile locking engagement between the parts is created to fix the shank head with respect to the receiver and retainer. The receiver and shank head retainer assemblies in the prior art have included a contractile retainer ring and/or a lower pressure insert with an expansion and contraction collet-type of structure having contractile locking engagement for the shank head due to direct contact between the retainer and/or the collet structure with the receiver resulting in contraction of the retainer ring and/or the collet-type structure of the insert against the shank head.

The prior art for modular polyaxial screw assemblies has also shown and taught that the contact surfaces on the outside of the collect and/or retainer and the inside of the receiver can be tapered, conical, radiused, spherical, curvate, multi-curvate, rounded, as well as other configurations to create a contractile type of locking engagement for the shank head with respect to the receiver.

In addition, the prior art for modular polyaxial screw assemblies has shown and taught that the shank head can both enter and escape from a collet-like structure on the insert or from the retainer when the insert or retainer is in the up position and within an expansion recess or chamber of the receiver. This is the case unless the insert and/or the retainer are blocked from being able to be pushed back up into receiver bore or cavity.

### SUMMARY OF THE INVENTION

The present invention differentiates from the prior art by not allowing the receiver to be removed from the shank head once the parts are connected. This is true even if the retainer can go back up into the expansion chamber. This approach or design has been found to be more secure and to provide more resistance to pull-out forces compared to the prior art for modular polyaxial screw designs. Collet-like structures extending downwardly from lower pressure inserts, when used in modular polyaxial screw designs, as shown in the prior art, have been found to be somewhat weak with respect to pull-out forces encountered during some spinal reduction procedures. Embodiments of the present invention are designed to solve such problems.

Embodiments of the present invention also differentiate from the prior art by providing a two- or multi-piece, retainer ring that is ultimately positioned in fixed relation with the shank, with most, if not all of the retainer ring positioned below the shank head hemisphere in the receiver, thus providing a stronger, more substantial structure to resist larger pull-out forces on the assembly. Furthermore, the retainer ring is also ultimately in sliding, pivoting relation with an inner surface of the receiver.

Thus, a polyaxial bone screw assembly according to an embodiment of the invention includes a shank having an integral upper portion illustrated as a spherical head and a body for fixation to a bone; a separate receiver defining an upper open channel, a central bore, a lower cavity and a lower opening; a compression insert; and a multi-piece interconnected retainer for capturing the shank head in the receiver lower cavity, the retainer being slidingly engageable with a surface defining the receiver cavity. In the illustrated embodiment, the shank upper portion or head is convex, more specifically, spherical, and the retainer is a two-piece interconnected structure having an inner concave surface, also illustrated with a spherical surface or surfaces. Illustrated tooling for locating the retainer about the spherical shank head within the receiver includes an inner plunger and an outer guide. The illustrated embodiment further includes an optional cooperating spring ring that seats in a groove of the spherical head and a groove of the retainer pieces. The retainer also has an outer convex surface, illustrated as spherical, and the receiver has an inner concave surface, illustrated as spherical, in slidable, pivoting and rotational relation thereto.

When assembled with the receiver, retainer and insert, but prior to locking, the shank head may be frictionally engaged with, but still movable in a non-floppy manner with respect to the illustrated lock and release insert to allow for movement of the shank to a desired position or angular orientation of the shank with respect to the receiver. The insert operatively engages the shank head and is spaced from the retainer by the shank head. The shank is finally locked into a fixed position relative to the receiver by frictional engagement between a portion of the insert due to a downward force placed on the compression insert by a closure top pressing on a rod, or other longitudinal connecting member, captured within the receiver bore and channel. In the illustrated embodiments, retainers and inserts are downloaded into the receiver, but uploaded retainer embodiments are also foreseen. The shank head can be positioned into the receiver lower cavity at the lower opening thereof prior to or after insertion of the shank into bone. As indicated above, some compression inserts may include a lock and release feature for independent locking of the polyaxial mechanism so the screw can be used like a fixed monoaxial screw. The shank can be cannulated for minimally invasive surgery applications.

In the illustrated embodiment, the ultimate locking of the shank between the compression insert and the retainer is the result of a locking expansion-type of contact between the shank head and the two-piece retainer and an expansion-type of non-tapered locking engagement between the retainer and an inner surface or surfaces of the receiver defining a lower portion of the receiver cavity. The shank head is forced down against the retainer during final locking. In some embodiments, when the polyaxial mechanism is locked, the insert is forced or wedged against surfaces of the receiver resulting in an interference, non-contractile locking engagement, allowing for adjustment or removal of the rod or other connecting member without loss of a desired angular relationship between the shank and the receiver. This independent, non-contractile locking feature allows the polyaxial screw to function like a fixed monoaxial screw.

The compression or pressure insert (a lock and release embodiment or a non-locking embodiment) may also be configured to be independently locked (permanently or temporarily) by a tool or instrument, thereby allowing the modular polyaxial screw to be distracted, compressed and/or rotated along and around the rod to provide for improved spinal correction techniques. Such a tool engages the receiver from the sides and then engages the insert to force the insert down into a locked position on the shank within the receiver. With the tool still in place and the correction maintained, the rod is then locked within the receiver channel by a closure top followed by removal of the tool. This process may involve multiple screws all being manipulated simultaneously with multiple tools to achieve the desired correction.

Objects of the invention further include providing apparatus and methods that are easy to use and especially adapted for the intended use thereof and wherein the tools are comparatively inexpensive to produce. Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is an exploded front elevational view of a polyaxial bone screw assembly according to an embodiment of the present invention including a shank, an open shank spring ring, a receiver, a two-piece retainer in an initial fixed orientation and a compression insert.

FIG. 2 is an enlarged top plan view of the shank of FIG. 1.

FIG. 3 is reduced cross-sectional view taken along the line 3-3 of FIG. 2.

FIG. 4 is an enlarged and partial perspective view of the shank and spring ring of FIG. 1 with portions broken away to show the detail thereof.

FIG. 5 is an enlarged perspective view of the receiver of FIG. 1.

FIG. 6 is a side elevational view of the receiver of FIG. 5.

FIG. 7 is a bottom plan view of the receiver of FIG. 5.

FIG. 8 is a top plan view of the receiver of FIG. 5.

FIG. 9 is a cross-sectional view taken along the line 9-9 of FIG. 8.

FIG. 10 is a cross-sectional view taken along the line 10-10 of FIG. 8.

FIG. 11 is an enlarged perspective view of the retainer of FIG. 1 having first and second pieces shown in an attached orientation.

FIG. 12 is a perspective view of the first piece of the two-piece retainer of FIG. 11.

FIG. 13 is an enlarged top plan view of the first retainer piece of FIG. 12.

FIG. 14 is an enlarged bottom plan view of the first retainer piece of FIG. 12.

FIG. 15 is a cross-sectional view taken along the line 15-15 of FIG. 13.

FIG. 16 is an enlarged perspective view of the second piece of the two-piece retainer of FIG. 11.

FIG. 17 is an enlarged top plan view of the second retainer piece of FIG. 16.

FIG. 18 is an enlarged bottom plan view of the second retainer piece of FIG. 16.

FIG. 19 is a cross-sectional view taken along the line 19-19 of FIG. 17.

FIG. 20 is an enlarged perspective view of the insert of FIG. 1.

FIG. 21 is a side elevational view of the insert of FIG. 20.

FIG. 22 is a front elevational view of the insert of FIG. 20.

FIG. 23 is a bottom plan view of the insert of FIG. 20.

FIG. 24 is a top plan view of the insert of FIG. 20.

FIG. 25 is a cross-sectional view taken along the line 25-25 of FIG. 24.

FIG. 26 is a cross-sectional view taken along the line 26-26 of FIG. 24.

FIG. 27 is an enlarged front elevational view of the retainer and receiver of FIG. 1 with portions of the receiver broken away to show the detail thereof and further showing in phantom an early stage of assembly of the retainer with the receiver.

FIG. 28 is an enlarged front elevational view with portions broken away, similar to FIG. 27, showing the retainer in a later stage of download and further showing the insert of FIG. 1 in enlarged side elevation, with an early stage of assembly of the insert with the receiver being shown in phantom.

FIG. 29 is a perspective view with portions broken away of the receiver, retainer and insert of FIG. 28, showing the insert rotated within the receiver during an assembly stage subsequent to that shown in FIG. 28 and further showing the subsequent step of crimping of the receiver against the insert.

FIG. 30 is a side elevational view of the assembly shown in FIG. 29.

FIG. 31 is a reduced front elevational view with portions broken away of the receiver, retainer and insert, similar to FIGS. 29 and 30 and further showing an assembly tool having an inner plunger and an outer guide, the tool positioned for entry into the receiver.

FIG. 32 is an enlarged and partial front elevational view of the assembly tool of FIG. 31 with portions broken away to show the detail thereof.

FIG. 33 is an enlarged and partial cross-sectional view taken along the line 33-33 of FIG. 32.

FIG. 34 is an enlarged and partial perspective view of the inner plunger of the assembly tool of FIG. 31.

FIG. 35 is an enlarged and partial front elevational view of the inner plunger of the assembly tool of FIG. 31.

FIG. 36 is an enlarged bottom plan view of the inner plunger of the assembly tool of FIG. 31.

FIG. 37 is an enlarged and partial front elevational view of the outer guide of the assembly tool of FIG. 31.

FIG. 38 is an enlarged and partial perspective view of the outer guide of the assembly tool of FIG. 31.

FIG. 39 is an enlarged and partial front elevational view with portions broken away of the assembly and tool of FIG. 31, the tool shown in an early stage of assembly with the receiver.

FIG. 40 is a partial front elevational view with portions broken away, similar to FIG. 39 with the outer guide of the assembly tool shown threaded with the receiver and further showing an alternative assembly stage with the shank of FIG. 1 shown in enlarged and partial front elevation in which the shank is first implanted in a vertebra, followed by assembly with the receiver, retainer and insert.

FIG. 41 is a partial front elevational view with portions broken away, similar to FIG. 40 showing the shank in an early stage of assembly with the retainer pieces.

FIG. 42 is a partial front elevational view with portions broken away, similar to FIG. 41, the retainer pieces being pushed up into engagement with the insert by the shank.

FIG. 43 is a partial front elevational view with portions broken away, similar to FIG. 42 showing a subsequent step of the shank spring ring being compressed inwardly into a groove in the shank by the retainer pieces.

FIG. 44 is a partial front elevational view with portions broken away, similar to FIG. 43 showing a subsequent step of the shank head being concentric with inner surfaces of the retainer pieces.

FIG. 45 is a partial perspective view with portions broken away showing a position of the plunger of the tool with respect to the retainer pieces wherein an adjustment (slight twist) of the tool may be required to place the plunger in a desired position for pressing down on the retainer pieces.

FIG. 46 is a partial front elevational view with portions broken away, similar to FIG. 44 showing a subsequent step of assembly that includes breaking a weld or other fixing between the retainer pieces to detach the pieces, the step performed by the plunger pressing upon the retainer pieces, and further showing an optional embodiment of the plunger having a guide pin for ensuring a desired axial alignment between the plunger, the retainer pieces and the shank.

FIG. 47 is a reduced and partial front elevational view with portions broken away, similar to FIG. 46 showing the retainer pieces pressed downwardly into a desired position about the shank head.

FIG. 48 is a partial front elevational view with portions broken away, similar to FIG. 47 showing a subsequent step of withdrawal of the plunger from the retainer and shank.

FIG. 49 is a partial front elevational view with portions broken away, similar to FIG. 48 showing a subsequent step of unscrewing the tool outer guide from the receiver.

FIG. 50 is a partial front elevational view of the assembly of FIG. 49 further shown in engagement with a rod and closure structure.

FIG. 51 is a partial side elevational view with portions broken away, showing the assembly of FIG. 50, but with the shank being articulated at an angle with respect to the receiver.

FIG. 52 is a partial perspective view with portions broken away, showing the shank, retainer, insert and receiver of FIG. 50 remaining in a locked position after removal of the rod and closure top (in exploded view).

FIG. 53 is a partial perspective view with portions broken away, similar to FIG. 52 and further showing, in exploded view, an alternative deformable rod and cooperating alternative closure top.

FIG. 54 is a partial front elevational view with portions broken away, showing the alternative rod and closure top fixed to the remainder of the assembly of FIG. 53.

FIG. 55 is a reduced and partial front elevational view with portions broken away of the assembly of FIG. 54 without the alternative rod and closure top, and further showing unlocking of the insert from the receiver with a two-piece tool having an inner insert engaging portion and an outer tubular holding portion.

FIG. 56 is a reduced and partial front elevational view of the two-piece tool of FIG. 55, holding prongs of the inner insert engaging portion being shown in phantom.

FIG. 57 is an enlarged and partial front elevational view of the inner insert engaging portion of the tool shown in FIG. 55 with portions broken away to show the detail thereof.

FIG. 58 is an enlarged and partial perspective view of an alternative assembly, similar to FIG. 1, shown with an alternative non-locking insert and further showing an alternative locking tool for independently locking such insert against the shank and thus locking the polyaxial movement of the assembly, even if a cooperating rod and closure top or in a loose unlocked relationship with the receiver or are removed.

FIG. 59 is an enlarged and partial perspective view of a portion of the locking tool of FIG. 58.

FIG. 60 is an enlarged front elevational view of the non-locking insert of FIG. 58.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. It is also noted that any reference to the words top, bottom, up and down, and the like, in this application refers to the alignment shown in the various drawings, as well as the normal connotations applied to such devices, and is not intended to restrict positioning of the bone attachment structures in actual use.

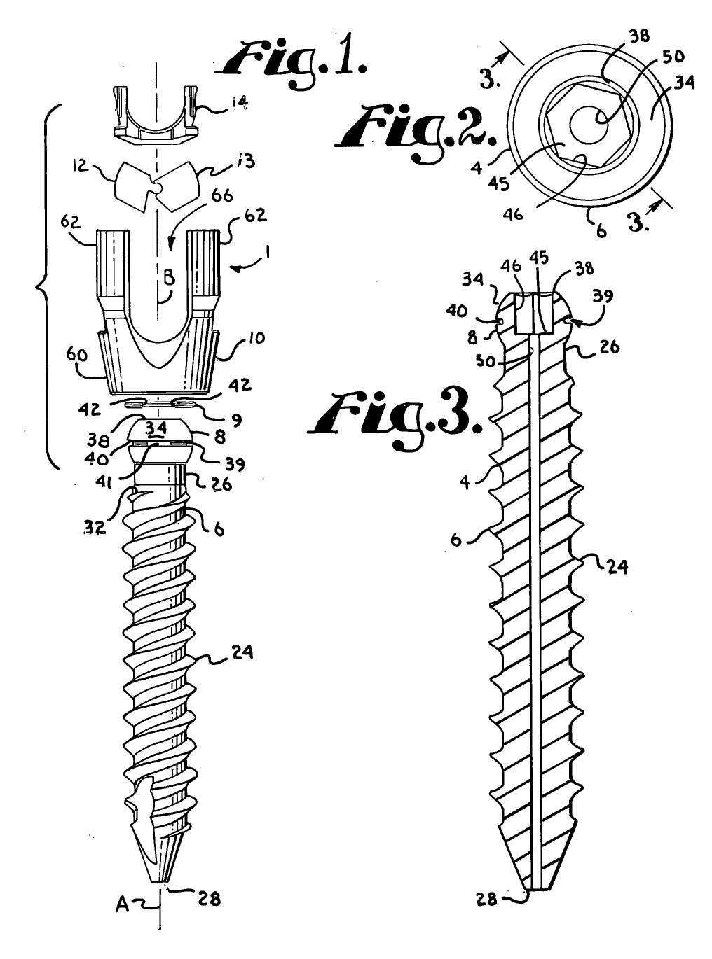

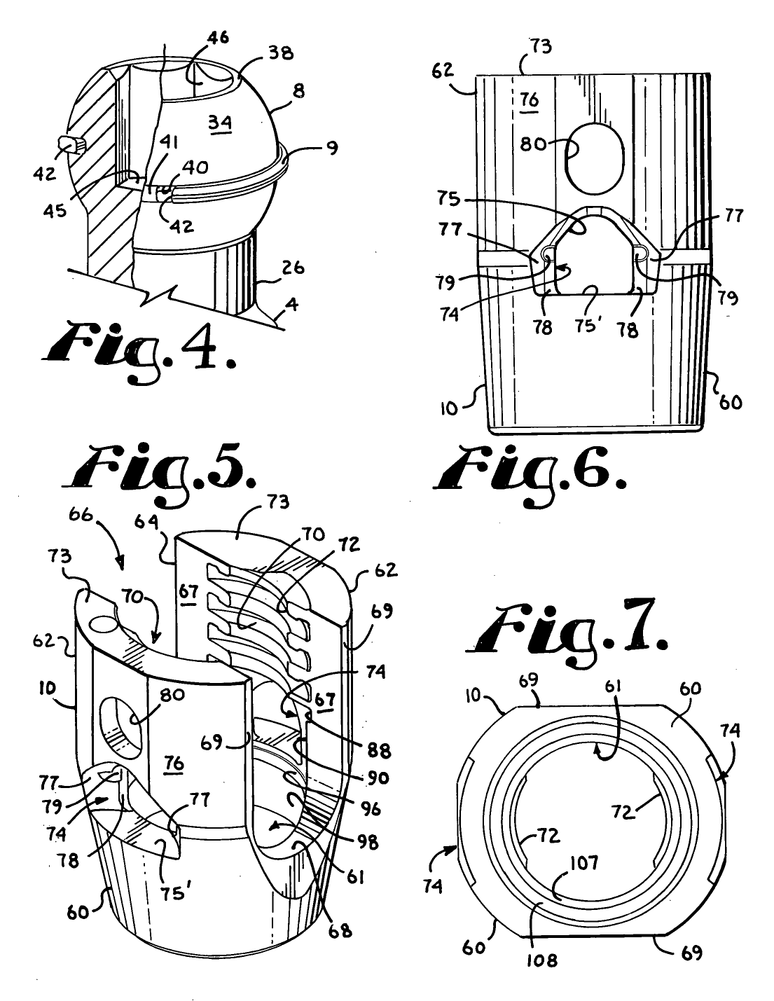

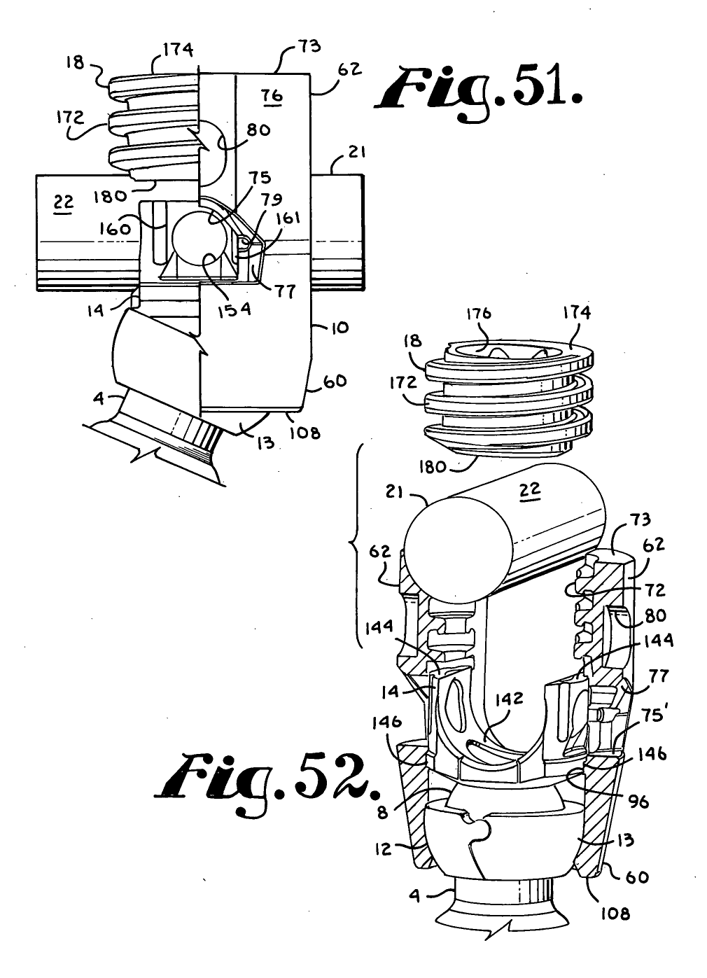

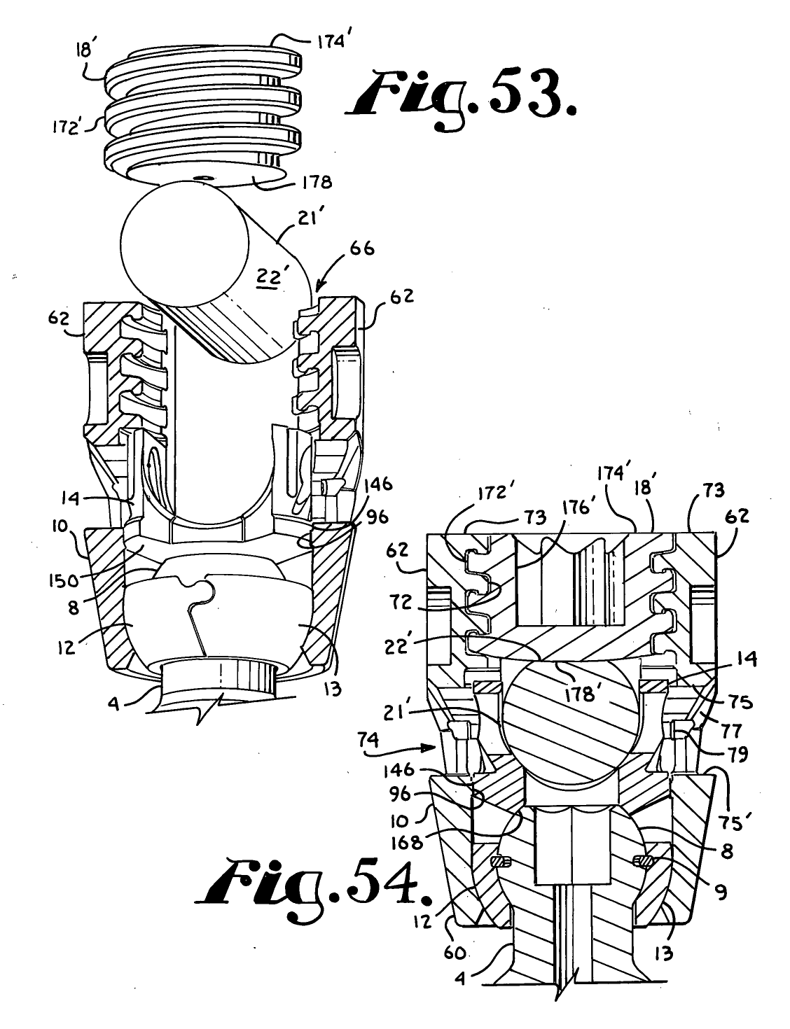

With reference to FIG. 1, the reference number 1 generally represents a polyaxial bone screw apparatus or assembly according to an embodiment of the present invention that includes a shank 4 that further includes a body 6 integral with an upwardly extending upper portion or head-like capture structure 8; an optional open or split annular spring ring 9; a receiver 10; a retainer structure illustrated as a two piece interconnected structure or ring having a first piece 12 and a second piece 13 initially welded or otherwise fixed to one another in a spacial relationship as shown in FIG. 1; and a compression or pressure insert 14. The receiver 10, retainer 12 and compression insert 14 are initially assembled and may be further assembled with the shank 4 either prior or subsequent to implantation of the shank body 6 into a vertebra 17 (see FIG. 40), as will be described in greater detail below. FIGS. 50-52 further show a closure structure 18 for capturing a longitudinal connecting member, for example, a rod 21 which in turn engages the compression insert 14 that presses against the shank upper portion 8 into fixed frictional contact with the retainer 12, so as to capture, and fix the longitudinal connecting member 21 within the receiver 10 and thus fix the member 21 relative to the vertebra 17. The illustrated rod 21 is hard, stiff, non-elastic and cylindrical, having an outer cylindrical surface 22. It is foreseen that in other embodiments, the rod 21 may be elastic, deformable and/or of a different cross-sectional geometry. The bone screw assembly 1 may also cooperate with soft connecting systems, such as spinal connectors having rigid sleeves for placement within the bone screw receiver in lieu of the rod 21, such sleeves including through bores for receiving a tensioned cord, for example. The receiver 10 and the shank 4 cooperate in such a manner that the receiver 10 and the shank 4 can be secured at any of a plurality of angles, articulations or rotational alignments relative to one another and within a selected range of angles both from side to side and from front to rear, to enable flexible or articulated engagement of the receiver 10 with the shank 4 until both are locked or fixed relative to each other near the end of an implantation procedure (see, e.g., FIG. 51 as compared to FIG. 52).

The shank 4, best illustrated in FIGS. 1-4, is elongate, with the shank body 6 having a helically wound bone implantable thread 24 (single or dual lead thread form) extending from near a neck 26 located adjacent to the upper portion or head 8, to a tip 28 of the body 6 and extending radially outwardly therefrom. During use, the body 6 utilizing the thread 24 for gripping and advancement is implanted into the vertebra 17 (e.g., see FIG. 40) leading with the tip 28 and driven down into the vertebra with an installation or driving tool (not shown), so as to be implanted in the vertebra to a location at or near the neck 26, as more fully described in the paragraphs below. The shank 4 has an elongate axis of rotation generally identified by the reference letter A.

The neck 26 extends axially upward from the shank body 6. The neck 26 may be of the same or of a slightly reduced radius as compared to an adjacent upper end or top 32 of the body 6 where the thread 24 terminates. Further extending axially and outwardly from the neck 26 is the shank upper portion or head 8 that provides a connective or capture apparatus disposed at a distance from the upper end 32 and thus at a distance from the vertebra 17 when the body 6 is implanted in such vertebra.

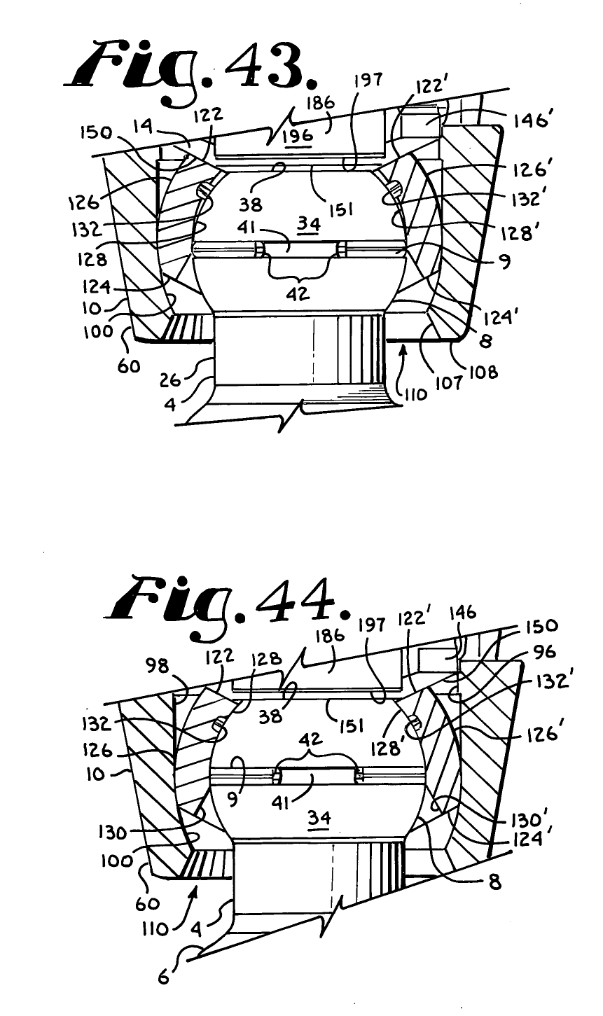

The shank upper portion 8 is configured for a fixed engagement between the portion 8 and the retainer pieces 12 and 13 and a pivotable connection between the shank 4 and the receiver 10 prior to fixing of the shank 4 in a desired position with respect to the receiver 10. The shank upper portion 8 has an outer, convex and substantially spherical surface 34 that extends outwardly and upwardly from the neck 26 and terminates at a substantially planar top or rim surface 38. The spherical surface 34 has an outer radius configured for frictional sliding and then ultimate fixed cooperation with a concave surface of the compression insert 14 and concave surfaces of the retainer pieces 13 and 14 as will be discussed more fully in the paragraphs below. The top surface 38 is substantially perpendicular to the axis A. The spherical surface 34 shown in the present embodiment is substantially smooth, but in some embodiments may include a roughening or other surface treatment. The shank spherical surface 34 is locked into place exclusively by the insert 14 and the retainer 12 and not by inner surfaces defining the receiver cavity, the shank being held in spaced relation with the receiver by the retainer 12. Formed at or adjacent a hemisphere of the surface 34 is a circumferential groove, generally 39 sized and shaped for receiving the spring ring 9. The illustrated groove 39 is defined in part by a segment 40 having a u-shaped profile that extends most of the way around the head 8 and a slightly raised segment or stop 41. The round, annular resilient spring ring 9, having a substantially circular profile and also being circular in cross-section, is located about the segment 40 but spaced radially outwardly therefrom when in a neutral state as shown, for example, in FIGS. 41 and 42. The ring 9 may have grooves formed therein, or have a roughened or other type of surface texture. The segment 41 provides a locator stop for open opposed ends 42 of the ring 9. When pressed into the groove 39, as shown for example in FIGS. 43 and 44, the ring 9 resiliently presses inwardly toward the surface 40 with the ends 42 located close to or in some instances abutting against segment 41. The resilient ring 9 then returns to a neutral or near neutral state when the retainer pieces 12 and 13 are moved into a fixed position with respect to the shank head 8 with the ring 9 ultimately being located partially in the groove 39 and partially in grooves formed in each of the pieces 12 and 13 as shown in FIG. 50, for example, and discussed in greater detail below, thus ensuring a desired alignment of the retainer pieces 12 and 13 with respect to the shank head 8. However, it is foreseen that in other embodiments of the invention, the spring ring 9 may not be utilized. Also, a stop or other protrusion on the shank head, for example, may be used in lieu of the spring ring.

A counter sunk substantially planar base 45 partially defines an internal drive feature or imprint 46. The illustrated internal drive feature 46 is an aperture formed in the top surface 38 and has a hex shape designed to receive a driving tool (not shown) of an Allen wrench type, into the aperture for rotating and driving the bone screw shank 4. It is foreseen that such an internal tool engagement structure may take a variety of tool-engaging forms and may include one or more apertures of various shapes, such as a pair of spaced apart apertures or a multi-lobular or star-shaped aperture, such as those sold under the trademark TORX, or the like. The seat or base surface 45 of the drive feature 46 is disposed substantially perpendicular to the axis A with the drive feature 46 otherwise being coaxial with the axis A. The drive seat 45 may include beveled or stepped surfaces that may further enhance gripping with the driving tool. In operation, a driving tool (not shown) is received in the internal drive feature 46, being seated at the base 45 and engaging the plurality of faces of the drive feature 46 for both driving and rotating the shank body 6 into the vertebra 17, either before the shank 4 is attached to the receiver 10 or after the shank 4 is attached to the receiver 10, with the shank body 6 being driven into the vertebra 17 with the driving tool extending into the receiver 10.

The shank 4 shown in the drawings is cannulated, having a small central bore 50 extending an entire length of the shank 4 along the axis A. The bore 50 is defined by an inner cylindrical wall of the shank 4 and has a circular opening at the shank tip 28 and an upper opening communicating with the external drive 46 at the driving seat 45. The bore 50 is coaxial with the threaded body 6 and the upper portion 8. The bore 50 provides a passage through the shank 4 interior for a length of wire (not shown) inserted into the vertebra 17 prior to the insertion of the shank body 6, the wire providing a guide for insertion of the shank body 6 into the vertebra 17.

To provide a biologically active interface with the bone, the threaded shank body 6 may be coated, perforated, made porous or otherwise treated. The treatment may include, but is not limited to a plasma spray coating or other type of coating of a metal or, for example, a calcium phosphate; or a roughening, perforation or indentation in the shank surface, such as by sputtering, sand blasting or acid etching, that allows for bony ingrowth or ongrowth. Certain metal coatings act as a scaffold for bone ingrowth. Bio-ceramic calcium phosphate coatings include, but are not limited to: alpha-tri-calcium phosphate and beta-tri-calcium phosphate (Ca₃(PO₄)₂, tetra-calcium phosphate (Ca₄P₂O₉), amorphous calcium phosphate and hydroxyapatite (Ca₁₀(PO₄)₆(OH)₂). Coating with hydroxyapatite, for example, is desirable as hydroxyapatite is chemically similar to bone with respect to mineral content and has been identified as being bioactive and thus not only supportive of bone ingrowth, but actively taking part in bone bonding.

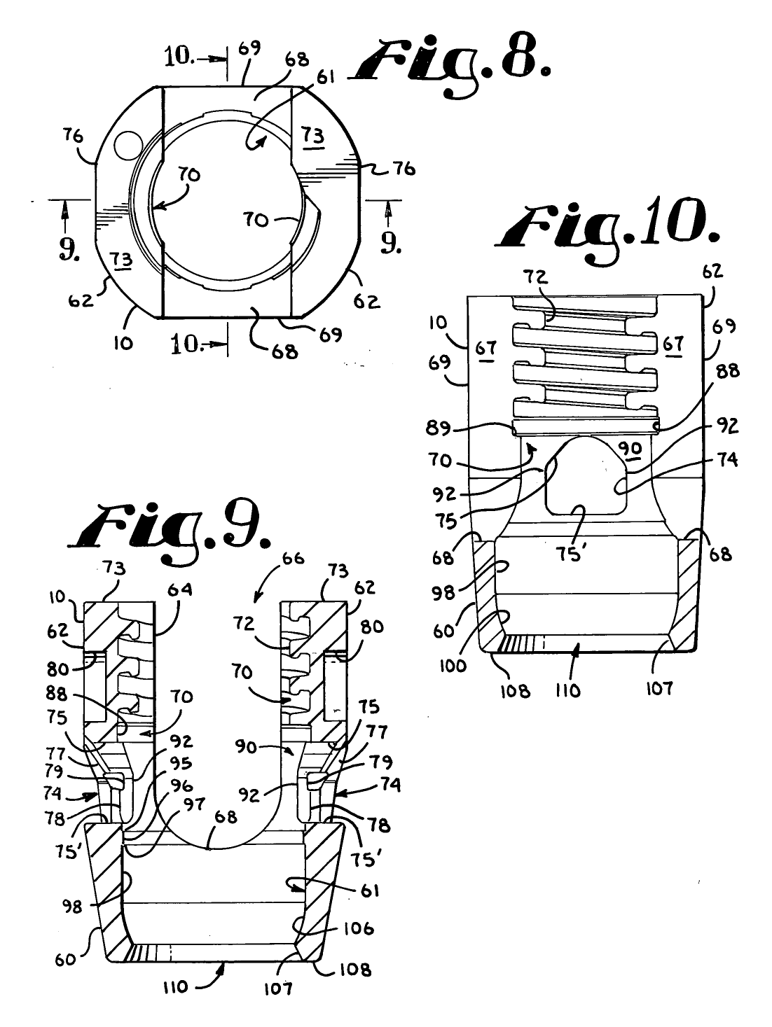

With particular reference to FIGS. 1 and 4-10, the receiver 10 has a generally U-shaped appearance with partially discontinuous and partially planar, frusto-conical and cylindrical inner and outer profiles. The receiver 10 has a central axis of rotation B that is shown in FIG. 1 as being aligned with and the same as the axis of rotation A of the shank 4, such orientation being desirable during assembly of the receiver 10 with the shank 4. After the receiver 10 is pivotally attached to the shank 4, either before or after the shank 4 is implanted in a vertebra 17, the axis B is typically disposed at an angle with respect to the axis A, as shown, for example, in FIG. 51.

The receiver 10 includes a base or lower body portion 60 that is illustrated as having a partially frusto-conical outer surface, that in some embodiments may include other outer surface geometries, including curved, cylindrical and partially planar. The base 60 defines a bore or inner cavity, generally 61, the base 60 being integral with a pair of opposed upstanding arms 62 forming a cradle and defining a channel 64 between the arms 62 with an upper opening, generally 66, the channel further defined by substantially planar arm surfaces 67 that extend downwardly to a U-shaped lower saddle or seat 68, the channel 64 having a width for operably snugly receiving the rod 21 or portion of another longitudinal connector between the arms 62; the channel 64 communicating with the base cavity 61. Outer front and rear opposed substantially planar arm surfaces 69 define an outer perimeter of the channel 64 at the arms 62 and about the channel seat 68.

Each of the arms 62 has an interior surface, generally 70, that includes various inner cylindrical profiles, an upper one of which is a partial helically wound guide and advancement structure 72 located adjacent substantially planar top surfaces 73 of each of the arms 62. In the illustrated embodiment, the guide and advancement structure 72 is a partial helically wound interlocking flangeform configured to mate under rotation with a similar structure on the closure structure 18, as described more fully below. However, it is foreseen that for certain embodiments of the invention, the guide and advancement structure 72 could alternatively be a square-shaped thread, a buttress thread, a reverse angle thread or other thread-like or non-thread-like helically wound discontinuous advancement structures, for operably guiding under rotation and advancing the closure structure 18 downward between the arms 62, as well as eventual torquing when the closure structure 18 abuts against the rod 21 or other longitudinal connecting member. It is foreseen that the arms could have break-off extensions.

An opposed pair of rounded off triangular or delta-shaped tool receiving and engaging apertures, generally 74, each having a through bore formed by an upper arched surface 75 and a substantially planar bottom surface 75′, are formed on outer surfaces 76 of the arms 62. The illustrated outer arm surfaces 76 include cylindrical and planar portions and may further include other curved surface portions. Each through bore defined by the surfaces 75 and 75′ extends through the arm to the inner surface 70. The apertures 74 with through bore portions 75 and 75′ are sized and shaped for receiving locking, unlocking and other manipulation tools and may aid in receiving and downloading the retainer ring 12 during top loading of the retainer 12 into the receiver 10. Each aperture 74 further includes a sloping tool alignment surface 77 that generally surrounds the arched bore portion 75 and does not extend completely through the respective arm 62, the sloping surfaces 77 terminating at a substantially planar thin wall 78 (or may be slightly curved), the wall 78 partly defining the bore portion 75 and disposed at an angle to the wall 78. Each wall 78 further includes a further recessed crimping portion or area 79 that is also partially formed in one of the sloping surfaces 77. As will be described in greater detail below, during an assembly stage, each of the four crimping portions 79 is pressed or crimped into the insert 14 to aid in retaining the insert 14 in alignment with the receiver and prohibit rotation of the insert with respect to the receiver, but to allow for some movement of the insert up and down along the receiver axis B. In other embodiments of the invention, other walls or surfaces defining the aperture 74 or other material defining other apertures or grooves may be inwardly crimped. It is noted that the illustrated receiver 10 is an integral structure and devoid of any spring tabs or collet-like structures. Alternatively, in some embodiments, spring tabs or other movable structure may be included on the receiver 10 or the insert 14 for retaining the insert 14 in a desired position, with regard to rotation and axial movement (along the axis A) with respect to the receiver 10. Preferably the insert and/or receiver are configured with structure for blocking rotation of the insert with respect to the receiver, but allowing some up and down movement of the insert with respect to the receiver during the assembly and implant procedure.

Formed in each surface 76 and located directly above the arched surface 75 and extending partially into each arm 62 is another tool receiving recess 80 having a somewhat circular or oval profile. Some or all of the apertures 74 and 80 may be used for holding the receiver 10 during assembly with the insert 14, the retainer 12 and the shank 4; during the implantation of the shank body 6 into a vertebra when the shank is pre-assembled with the receiver 10; during assembly of the bone anchor assembly 1 with the rod 21 and the closure structure 18; and during lock and release adjustment of the some inserts with respect to the receiver 10, either into or out of frictional engagement with the inner surfaces of the receiver 10 as will be described in greater detail below. It is foreseen that tool receiving grooves, depressions or apertures may be configured in a variety of shapes and sizes and be disposed at other locations on the receiver arms 62.

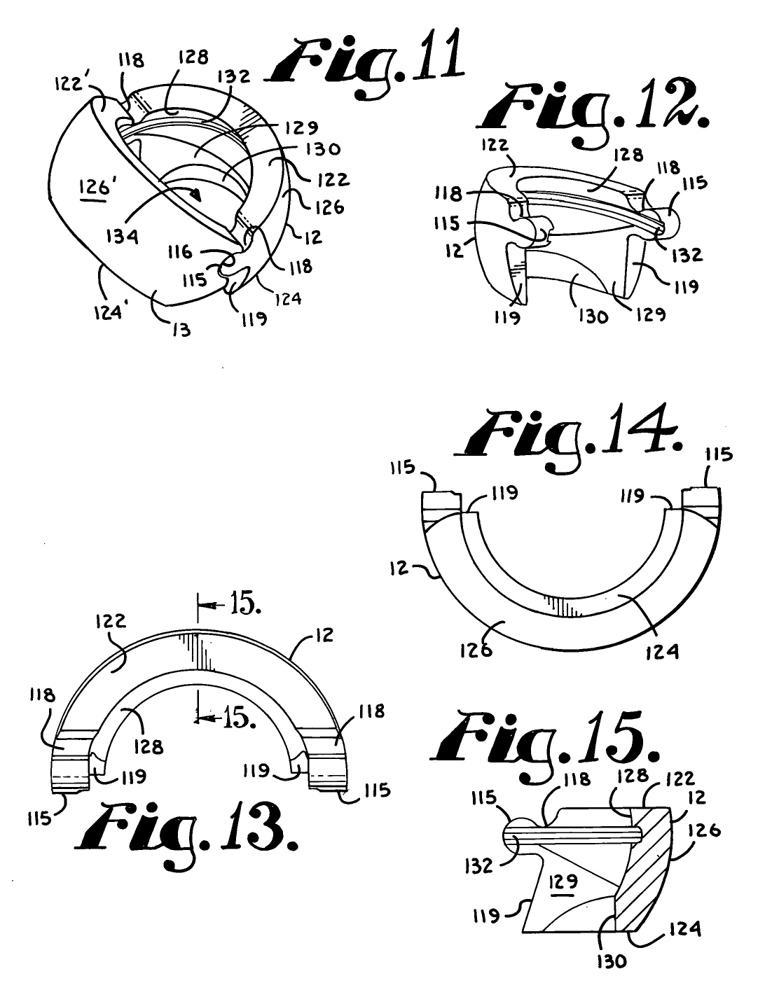

Returning to the interior surface 70 of the receiver arms 62, located below the guide and advancement structure 72 is a discontinuous cylindrical surface 88 partially defining a run-out feature for the guide and advancement structure 72. The cylindrical surface 88 has a diameter equal to or slightly greater than a greater diameter of the guide and advancement structure 72. Moving downwardly in a direction toward the base 60, adjacent the cylindrical surface 88 of each arm is a run-out seat or surface 89 that extends inwardly toward the axis B and gently slopes downwardly toward the axis B. In some embodiments, the surface 89 may be perpendicular to the axis B. Adjacent to and located below the surface 89 is another cylindrical surface 90 having a diameter smaller than the diameter of the surface 88. The through bore surfaces 75 and 75′ extend through the arms at the surfaces 90. In some embodiments an upper portion of each arch 75 may extend through the surfaces 88. Located near each aperture surface 75 is an inner surface portion 92 of the crimp areas or portions 79, the surface portions 92 engaging the insert 14 when the thin wall at the surface portion 79 is crimped toward the insert 14 during assembly of such insert in the receiver 10 as will be described in greater detail below. With particular reference to FIG. 9, the inner discontinuous surface 90 found on the receiver arms 62 also extends slightly downwardly into the receiver cavity 61. The surface 90 is disposed parallel to the receiver axis B and is sized to receive portions of the insert 14. The surface 90 terminates at a stepped surface 95 that extends inwardly toward the axis B. Adjacent the surface 95 is a narrow cylindrical band 96 having a diameter slightly smaller than a diameter of the surface 90. The band 96 is sized to provide a locking interference fit with a cylindrical portion of the locking insert 14 as will be described in greater detail below. Below and adjacent the band 96 is a ledge 97 that in turn is adjacent to a cylindrical surface 98 having a diameter slightly greater than the diameter of the cylindrical band 96. The surface 98 defines a large working portion of the cylindrical cavity 61 in which the two retainer pieces 12 and 13 are loaded and manipulated during assembly with the bone screw shank head or upper portion 8. Adjacent and below the cylindrical surface 98 is an inner spherical surface 100 sized and shaped for sliding relation and ultimate frictional contact with an outer surface of each of the retainer pieces 12 and 13 as will be described in greater detail below. Located below and adjacent to the surface 100 is a beveled or flared bottom opening surface 107, the surface 107 communicating with an exterior base surface 108 of the base 60, defining a lower opening, generally 110, into the base cavity 61 of the receiver 10.

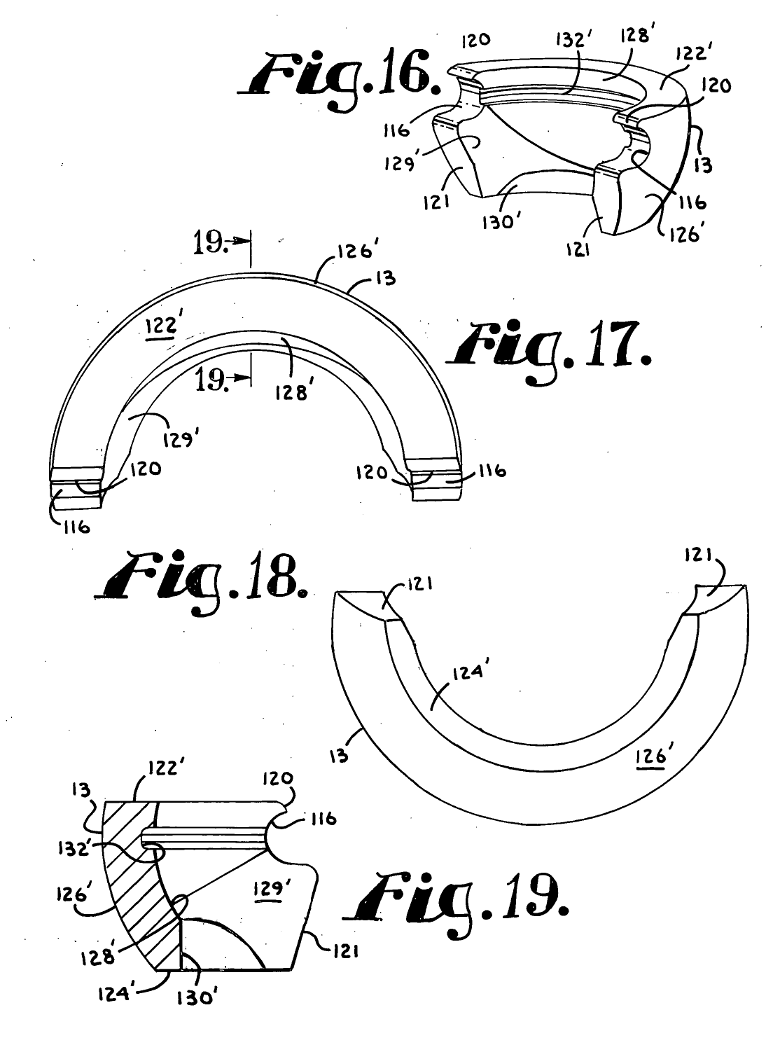

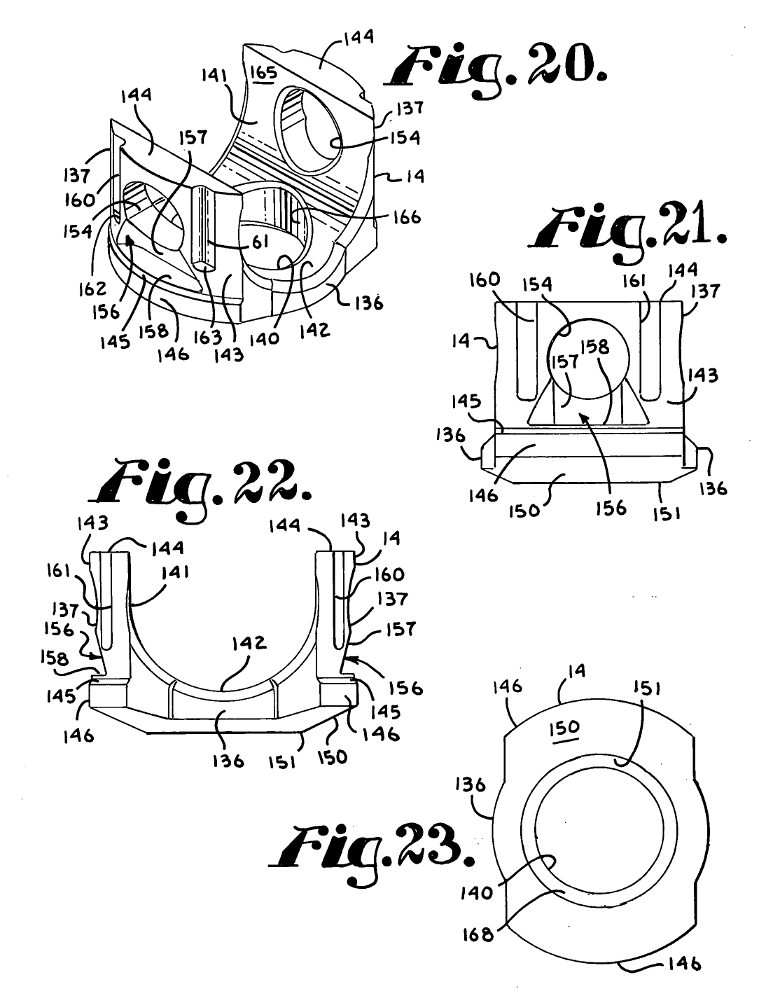

With particular reference to FIGS. 1 and 11-19, the retainer ring pieces 12 and 13, are initially fixed to one another by a weld, adhesive, or other temporary fixing means, to provide a shape easily top loadable through the opening 66 and into the cavity 61 of the receiver 10. Then, during assembly with the shank head 8, the weld or other fixing between the pieces 12 and 13 is broken and the pieces 12 and 13 are pivoted with respect to one another to a position that captures the shank upper portion 8 within the receiver 10, the resulting interconnected ring 12,13 surrounding the shank head 8 and sharing the central axis A with the shank 4, with the axis 4 ultimately operationally being the same or different than the axis B associated with the receiver 10. The resulting interconnected two-piece retainer ring 12,13 is articulatable and slidable with respect to the receiver 10 until locked into place. The retainer ring pieces are typically made from a strong, hard material, such as a stainless steel or titanium alloy, so that the retainer pieces 12,13 may be manipulated during various steps of assembly and provide a strong, pull-out resistant component as will be described in greater detail below. The pieces 12 and 13 are substantially similar to one another with the exception of end portions in which the piece 12 includes two jig-saw-puzzle-like knobs 115 and the piece 13 includes two cooperating jig-saw-puzzle-like grooves or apertures 116 sized and shaped for receiving the knobs 115. The piece 12 further includes an upper curved groove or depression 118 located adjacent each knob 12 sized and shaped for sliding cooperation with and temporary attachment of the piece 12 with the piece 13 in a hinged or folded geometry shown in FIG. 11 that is configured for loading into the receiver 10 as shown, for example, in FIGS. 27 and 28 as well as an initial receiving engagement with the shank head 8, as shown, for example, in FIG. 41. The retainer piece 12 further includes a pair of lower end surfaces 119 extending downwardly from the knob 115. The retainer piece 13 includes a pair of upper lips 120 for sliding and pivoting close to the retainer 12 depressions 118 and a pair of lower end surfaces 121 for close or touching cooperation with the retainer 12 side surfaces 119 when the pieces 12 and 13 are in a final operational orientation surrounding the shank head 8. When in an operation orientation with the shank head 8, the pieces 12 and 13 are still linked by the knobs 115 and cooperating grooves 116, but there is some space in and around each knob 115 allowing for the pieces 12 and 13 to ultimately move slightly away from each other when a locking force is applied to the shank head 8 from above, the pieces 12 and 13 being pressed outwardly into locked frictional engagement with the receiver inner surface 100. Other than the end portion surfaces making up and located above and below the retainer 12 knob 12 and the retainer 13 groove 13, the pieces 12 and 13 include substantially similar surface features, including respective planar top surfaces 122 and 122′, planar bottom surfaces 124 and 124′, outer convex spherical surfaces 126 and 126′ located between the respective top and bottom surfaces, inner concave spherical surfaces 128 and 128′, inner concave spherical surfaces 129 and 129′ and inner cylindrical surfaces 130 and 130′. During loading of the retainer when the pieces 12 and 13 are welded or otherwise fixed to one another at the knobs 115 and the surfaces making up the groove 116, the top surfaces 122 and 122′ and the bottom surfaces 126 and 126′ are disposed at an angle to each other. After the weld or other fixing means is broken and the knobs 115 are pivoted with respect to the grooves 116, the top surfaces 122 and 122′ are in substantially the same first plane and the bottom surfaces 124 and 124′ are in the substantially same second plane, the first and second planes being parallel to one another. The convex outer radiused surfaces 126 and 126′ are both sized and shaped for sliding and then ultimate frictional engagement with the receiver inner radiused surface 100, the surfaces 126 and 126′ having a radius that is the same or substantially similar to the radius of the surface 100. The concave inner radiused surfaces 128 and 128′ each have a radius that is the same or substantially similar to a radius of the shank head 8 convex outer surface 34. The concave inner surfaces 129 and 129′ each have a radius that is larger than the radius of the shank surface 34, allowing for the pieces 12 and 13 to readily pivot about the shank head 8 during assembly of the pieces 12 and 13 about the head 8. The cylindrical surfaces 130 and 130′ are sized and shaped to closely fit about the cylindrical surface of the shank neck 26. The inner substantially spherical surfaces 128 and 128′ each have a respective groove 132 and 132′ located spaced from and running parallel to the respective top surfaces 122 and 122′. With respect to the retainer piece 12, the groove 132 runs between the knobs 115 and terminates at outer surfaces thereof. With respect to the retainer piece 13, the groove 132′ runs between the grooves 116 at a location below the lips 120. The grooves 132 and 132′ are ultimately aligned in the same plane and are sized to receive an outer surface of the spring ring 9 that is partially located in the U-shaped portion 40 of the groove 39. The retainer pieces 12 and 13 form a central channel or through bore, generally 134, that passes entirely through the ring pieces 12 and 13 from the respective top surfaces 122 and 122′ to the respective bottom surfaces 124 and 124′ thereof.

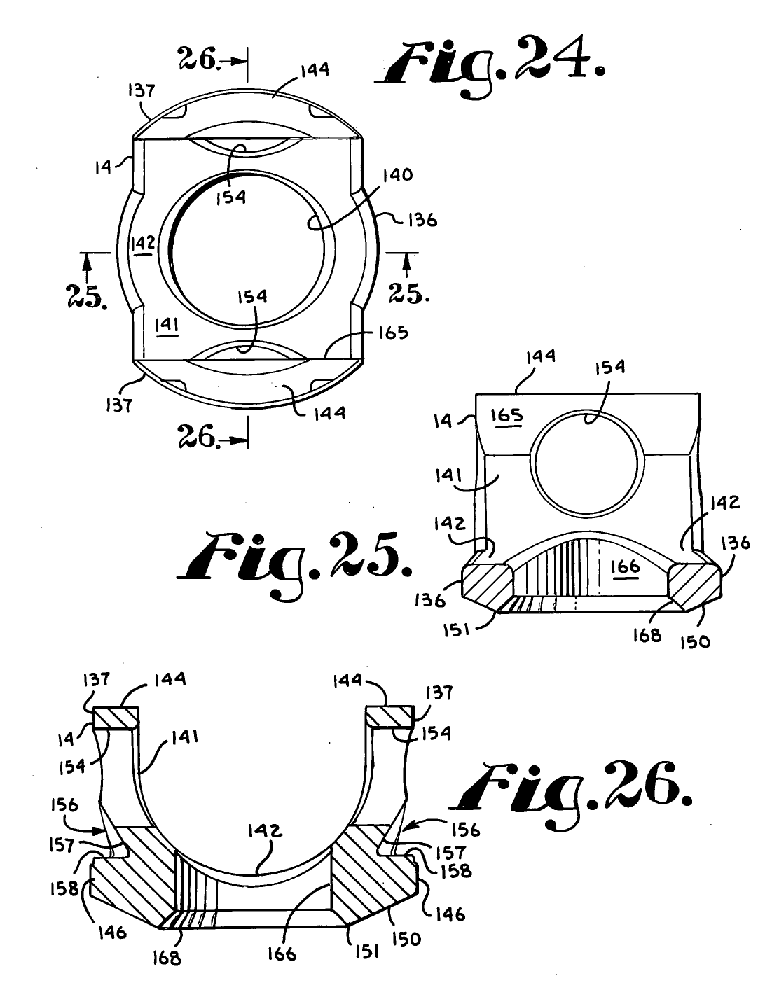

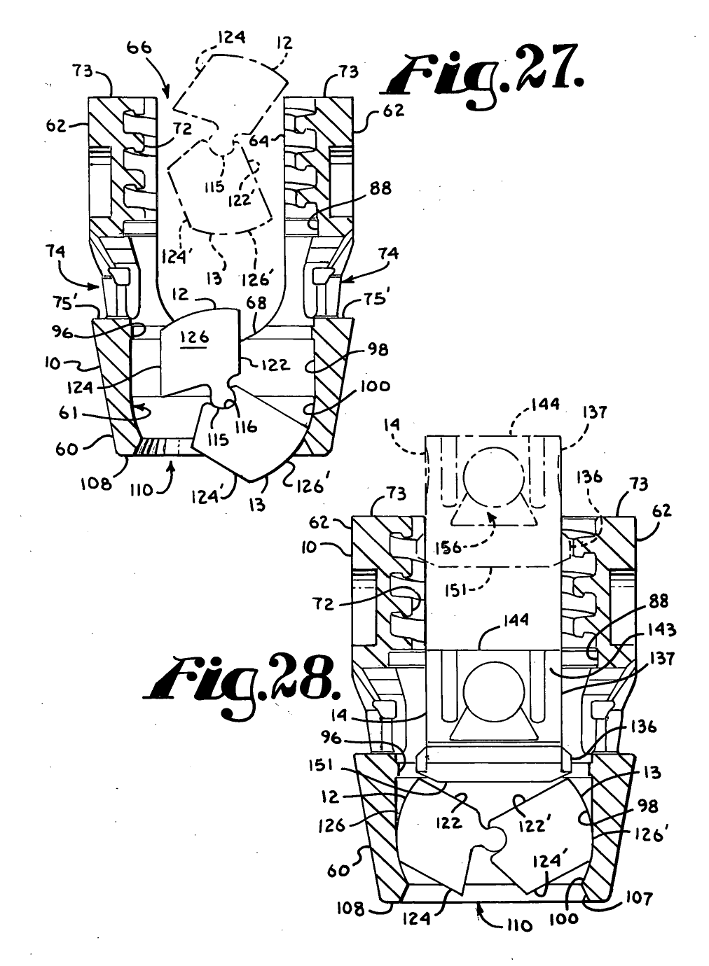

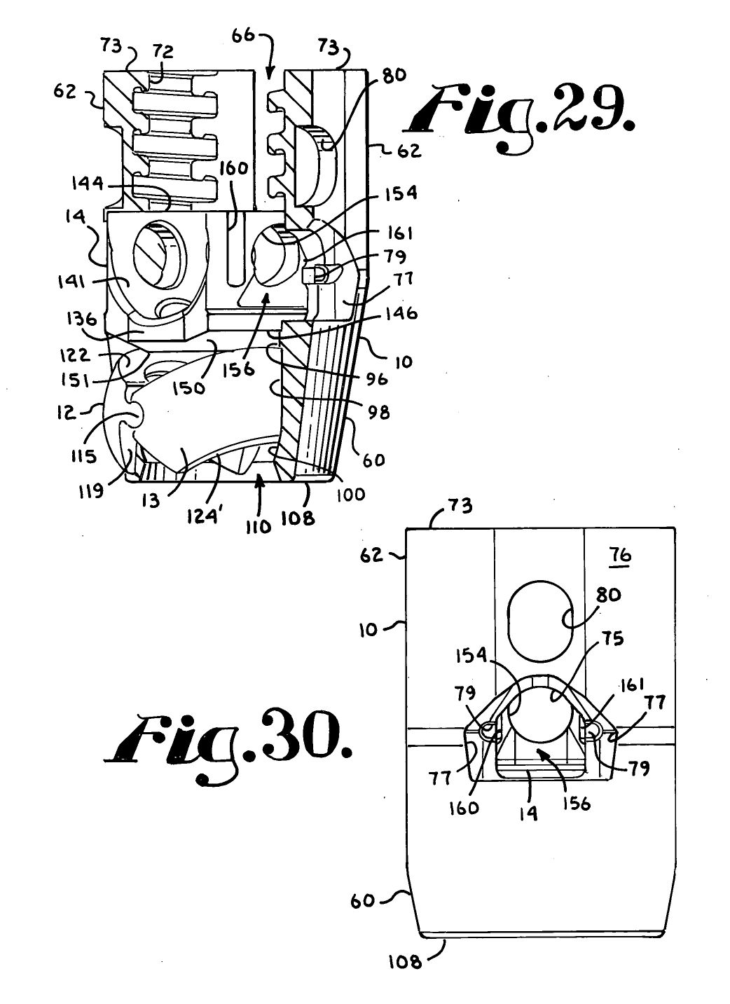

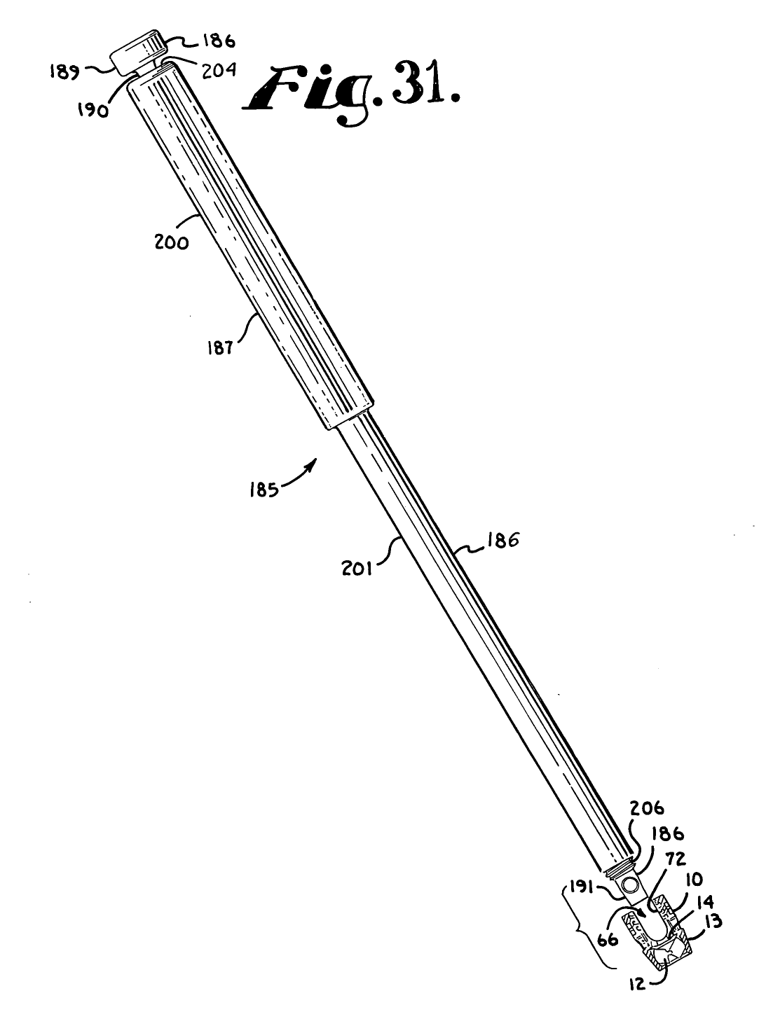

With particular reference to FIGS. 1 and 20-26, the crown compression insert 14 is illustrated that is sized and shaped to be received by and down-loaded into the receiver 10 at the upper opening 66. The compression insert 14 has an operational central axis that is the same as the central axis B of the receiver 10. In operation, the lock and release insert 14 may be advantageously manipulated downwardly into a friction or interference fit with the receiver wherein the insert 14 frictionally engages the bone screw shank upper portion 8, but is not locked against the portion 8, (i.e., movement occurs when some force is applied) allowing for a non-floppy movement and placement of the shank 4 with respect to the receiver 10 at a desired angle during surgery prior to locking of the shank with respect to the receiver near the end of the procedure. In the illustrated embodiment, the inert 14 is forced into an interference fit engagement with the receiver 10 at the inner cylindrical surface 96, and thus is capable of retaining the shank 6 in a locked position even if the rod 21 and closure top 18 are removed. Such locked position may also be released by the surgeon if desired. The insert 14 (as well as an alternative non-locking insert 14′ shown in FIG. 60) is preferably made from a solid resilient material, such as a stainless steel or titanium alloy, so that portions of the insert may be snapped or popped onto the shank upper portion 8 as well as pinched or pressed against and un-wedged (in certain embodiments) from the receiver 10 with a release tool.

The locking compression insert 14 includes a substantially cylindrical body 136 integral with a pair of upstanding arms 137. A bore, generally 140, is disposed primarily within and through the body 136 and communicates with a generally U-shaped through channel formed by a saddle 141 that is partially defined by the upstanding arms 137 and partially by the body 136. The saddle 141 is sized and shaped to closely, snugly engage the cylindrical rod 21 and includes a curved lower seat 142. Upper portions of the saddle 141 located near top surfaces of each arm 137 are substantially planar. It is foreseen that an alternative embodiment may be configured to include planar holding surfaces that closely hold a square or rectangular bar as well as hold a cylindrical rod-shaped, cord, or sleeved cord longitudinal connecting member. The arms 137 that are substantially cylindrical in outer profile are sized and configured for ultimate placement at or near the cylindrical run-out surface 88 and inner surface 90 located below the receiver guide and advancement structure 72. It is foreseen that in some embodiments of the invention, the insert arms 137 may be extended upwardly and the closure top configured such the arms ultimately directly engage the closure top for locking of the polyaxial mechanism, for example, when the rod 21 is made from a deformable material. In such embodiments, the insert 14 would include a rotation blocking structure or feature on an outer surface thereof that abuts against cooperating structure located on an inner wall of the receiver 10, preventing rotation of the insert with respect to the receiver when the closure top is rotated into engagement with the insert.

In the present embodiment, each of the arms 137 includes an outer surface 143 that is illustrated as partially cylindrical and runs from the substantially planar top surfaces 144 to an outwardly and downwardly sloping ledge 145 that in turn is adjacent to another partially cylindrical surface 146 having a diameter greater than the surface 143. The surface 146 is sized and shaped for interference locking fit with the inner cylindrical band 96 of the receiver 10. The surface 146 is adjacent to an inwardly sloping lower surface 150 of the insert 14, the surface 150 extending about the body 136 and the arms 137 and terminating at an annular bottom surface 151. The surface 150 is advantageously sloped or angled, running from the lower edge or rim 151 outwardly and upwardly away from the axis B and toward the upper surfaces 144, this allows the surface 150 to cooperate with and engage the retainer 12 and 13 top surfaces 122 and 122′ when the retainer pieces and shank 4 are being assembled within the receiver 10. The sloping surface 150 also provides clearance between the fully assembled retainer pieces 12 and 13 and the insert 14 when the shank 4 and attached retainer pieces 12 and 13 are articulated or pivoted with respect to the receiver 10.

The surfaces 143 are sized and shaped to generally fit within the receiver arms 62. The arm outer surfaces 143 further include notches or grooves formed thereon for receiving manipulation, unlocking and locking tools. Although not shown, each surface 143 may include one or more through bores or other apertures for receiving tooling, particularly useful for alternative locking embodiments (not shown). Centrally located below a circular through bore 154 is a delta or triangular notch or recess, generally 156, for receiving tooling defined in part by an upper sloping surface 157 and intersecting a lower planar surface 158 disposed substantially perpendicular to a central axis of the insert 14 (and the axis B of the receiver when the insert is disposed within the receiver). Each of the surfaces 157 and surface 158 cooperate and align with the respective receiver aperture through bore surfaces 77 and 75′ when the insert 14 is captured and operationally positioned within the receiver 10 as will be described in greater detail below. In the illustrated embodiments, also formed in each surface 143 are a pair of spaced v- or squared-off notches or grooves 160 and 161 that run from the respective top surface 144 to near the sloping surface 157 of the central delta cut or notch 156. The grooves 160 and 161 cooperate with the receiver crimp wall 79 inner surfaces 92 to aid in alignment of the insert channel saddle 141 with the receiver channel 64 as shown, for example in FIG. 29. The illustrated pair of grooves 160 and 161 are disposed substantially parallel to the central axis of the insert 14, running from one of the top surfaces 144 to respective lower or bottom surfaces 162 and 163.

The u-shaped channel formed by the saddle 141 is also partially defined by opposed inner planar surfaces 165 located near the arm top surfaces 144. The saddle 141 also communicates with the bore 140 at an inner cylindrical surface 166, the surface 166 located centrally within the insert body 136 and further communicating with a lower concave surface portion 168 having a generally spherical profile with a radius the same or substantially similar to a radius of the surface 34 of the shank upper portion or head 8. The surface 168 terminates at the edge or rim 151. It is foreseen that in some embodiments of the invention a portion or all of the surface 168 may include ridges, stepped surfaces or a surface roughening or texture, such as scoring or knurling, or the like, for enhancing frictional engagement with the shank upper portion 8.

The insert bore 140 is sized and shaped to receive the driving tool (not shown) therethrough that engages the shank drive feature 46 when the shank body 6 is driven into bone with the receiver 10 attached. Also, the bore 140 may receive a manipulation tool used for releasing the such insert from a locked position with the receiver, the tool pressing down on the shank and also gripping the insert at the opposed through bores or with other tool engaging features. A manipulation tool for un-wedging a locking insert from the receiver 10 may also access the such tooling bores from the receiver through bores 74. The illustrated insert 14 may further include other features, including grooves and recesses for manipulating and holding the insert 14 within the receiver 10 and providing adequate clearance between the retainer 12 and the insert 14.

The insert body 136 located between the arms 137 has an outer diameter slightly smaller than a diameter between crests of the guide and advancement structure 72 of the receiver 10, allowing for top loading of the compression insert 14 into the receiver opening 66, with the arms 137 of the insert 14 being located between the receiver arms 62 during insertion of the insert 14 into the receiver 10. Once the arms 137 of the insert 14 are generally located beneath the guide and advancement structure 72, the insert 14 is rotated into place about the receiver axis B until the top surfaces 144 are located directly below the guide and advancement structure 72 as will be described in greater detail below.

With reference to FIGS. 50-52, for example, the illustrated elongate rod or longitudinal connecting member 21 (of which only a portion has been shown) can be any of a variety of implants utilized in reconstructive spinal surgery, but is typically a cylindrical, elongate structure having the outer substantially smooth, cylindrical surface 22 of uniform diameter. The rod 21 may be made from a variety of metals, including hard and soft metal alloys and hard and soft or deformable and less compressible plastics, including, but not limited to rods made of elastomeric, polyetheretherketone (PEEK) and other types of materials.

Longitudinal connecting members for use with the assembly 1 may take a variety of shapes, including but not limited to rods or bars of oval, rectangular or other curved or polygonal cross-section. The shape of the insert 14 may be modified so as to closely hold, and if desired, fix or slidingly capture the longitudinal connecting member to the assembly 1. Some embodiments of the assembly 1 may also be used with a tensioned cord. Such a cord may be made from a variety of materials, including polyester or other plastic fibers, strands or threads, such as polyethylene-terephthalate. Furthermore, the longitudinal connector may be a component of a longer overall dynamic stabilization connecting member, with cylindrical or bar-shaped portions sized and shaped for being received by the compression insert 14 of the receiver having a U-shaped, rectangular- or other-shaped channel, for closely receiving the longitudinal connecting member. The longitudinal connecting member may be integral or otherwise fixed to a bendable or damping component that is sized and shaped to be located between adjacent pairs of bone screw assemblies 1, for example. A damping component or bumper may be attached to the longitudinal connecting member at one or both sides of the bone screw assembly 1. A rod or bar (or rod or bar component) of a longitudinal connecting member may be made of a variety of materials ranging from soft deformable plastics to hard metals, depending upon the desired application. Thus, bars and rods may be made of materials including, but not limited to metal and metal alloys including but not limited to stainless steel, titanium, titanium alloys and cobalt chrome; or other suitable materials, including plastic polymers such as polyetheretherketone (PEEK), ultra-high-molecular weight-polyethylene (UHMWP), polyurethanes and composites, including composites containing carbon fiber, natural or synthetic elastomers such as polyisoprene (natural rubber), and synthetic polymers, copolymers, and thermoplastic elastomers, for example, polyurethane elastomers such as polycarbonate-urethane elastomers.

With reference to FIGS. 50-52, the closure structure or closure top 18 shown with the assembly 1 is rotatably received between the spaced arms 62 of the receiver 10. It is noted that the closure 18 top could be a twist-in or slide-in closure structure. The illustrated closure structure 18 is substantially cylindrical and includes a an outer helically wound guide and advancement structure 172 in the form of a flange that operably joins with the guide and advancement structure 72 disposed on the arms 62 of the receiver 10. The flange form utilized in accordance with embodiments of the present invention may take a variety of forms, including those described in Applicant's U.S. Pat. No. 6,726,689, which is incorporated herein by reference. Although it is foreseen that the closure structure guide and advancement structure could alternatively be a buttress thread, a square thread, a reverse angle thread or other thread like or non-thread like helically wound advancement structure, for operably guiding under rotation and advancing the closure structure 18 downward between the arms 62 and having such a nature as to resist splaying of the arms 62 when the closure structure 18 is advanced into the channel 64, the flange form illustrated herein as described more fully in Applicant's U.S. Pat. No. 6,726,689 is preferred as the added strength provided by such flange form beneficially cooperates with and counters any reduction in strength caused by the inset surfaces 69 resulting in a reduced profile of the illustrated receiver 10 at the U-shape channel, such surfaces advantageously engaging longitudinal connecting member components as will be further described below. The illustrated closure structure 18 also includes a top surface 174 with an internal drive 176 in the form of an aperture that is illustrated as a star-shaped internal drive such as that sold under the trademark TORX, or may be, for example, a hex drive, or other internal drives such as slotted, tri-wing, spanner, two or more apertures of various shapes, and the like. A driving tool (not shown) sized and shaped for engagement with the internal drive 176 is used for both rotatable engagement and, if needed, disengagement of the closure 18 from the receiver arms 62. It is also foreseen that the closure structure 18 may alternatively include a break-off head designed to allow such a head to break from a base of the closure at a preselected torque, for example, 70 to 140 inch pounds. Such a closure structure would also include a base having an internal drive to be used for closure removal. A base or bottom surface 178 of the closure is planar and further includes a rim 180 and may or may not include a further include a central point 181, the rim 180 and or the point 181 for engagement and penetration into the surface 22 of the rod 21 in certain embodiments of the invention. The closure top 18 may further include a cannulation through bore (not shown) extending along a central axis thereof and through the top and bottom surfaces thereof. Such a through bore provides a passage through the closure 18 interior for a length of wire (not shown) inserted therein to provide a guide for insertion of the closure top into the receiver arms 62.

With reference to FIGS. 53 and 54, an alternative closure top 18′ is shown with an alternative deformable rod 18′, the closure top 18′ substantially identical to the closure top 18 with the exception that a radiused bottom surface 178′ replaces the bottom surface 178 with a rim 180 and point 181. Otherwise, the closure top 18′ includes a guide and advancement structure 172′, a top surface 174′, and an internal drive 176′ that are the same or similar in form and function to the respective guide and advancement structure 172, top surface 174, and internal drive 176 previously discussed herein with respect to the closure top 18.

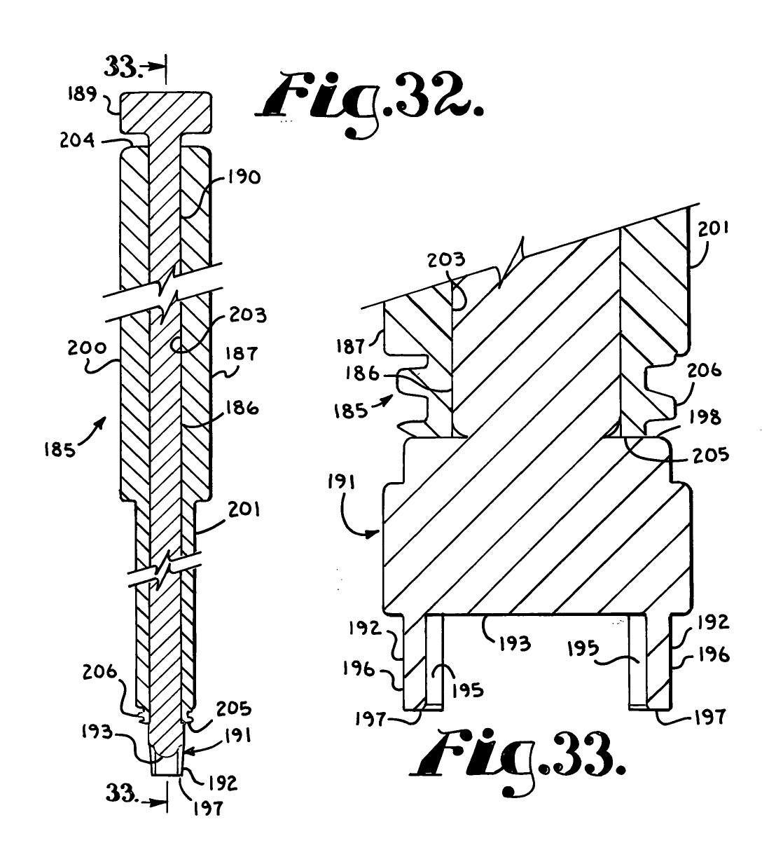

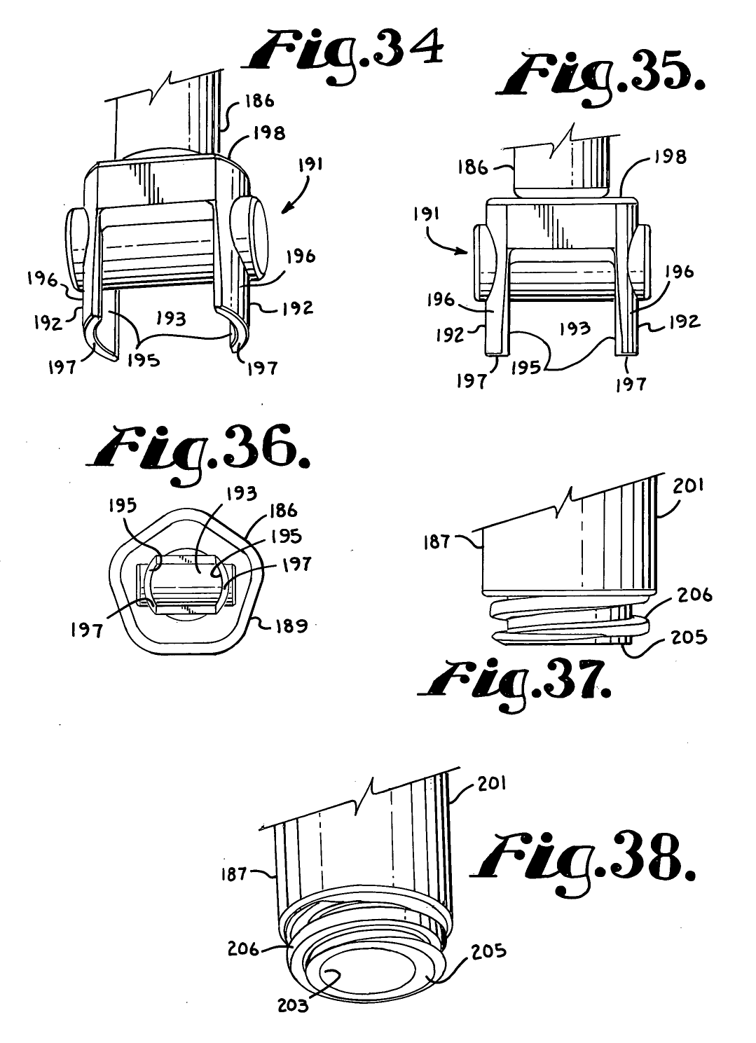

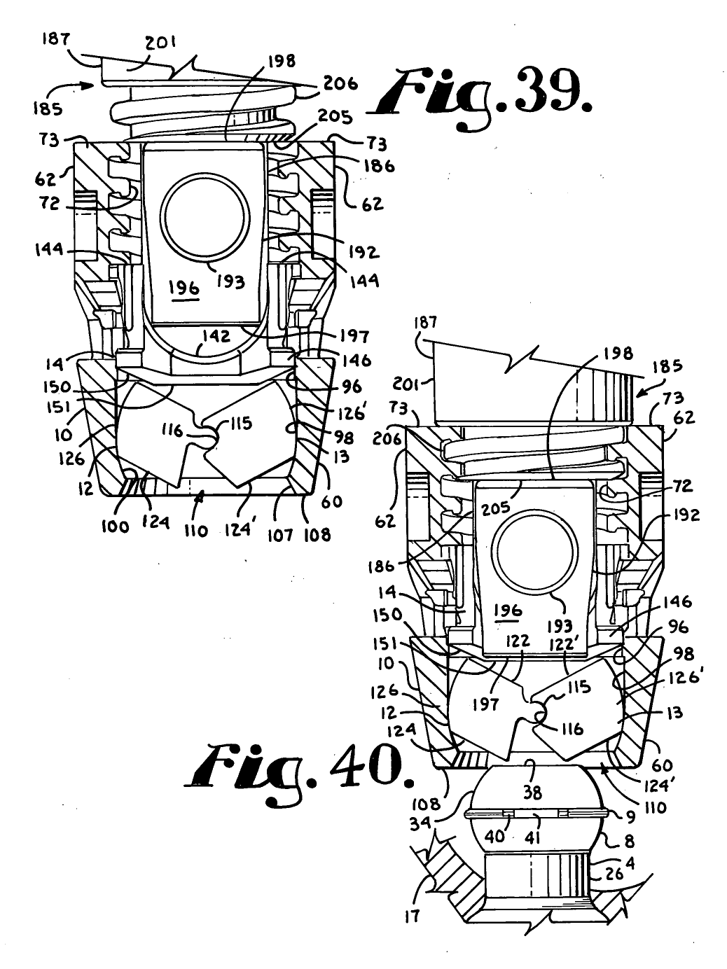

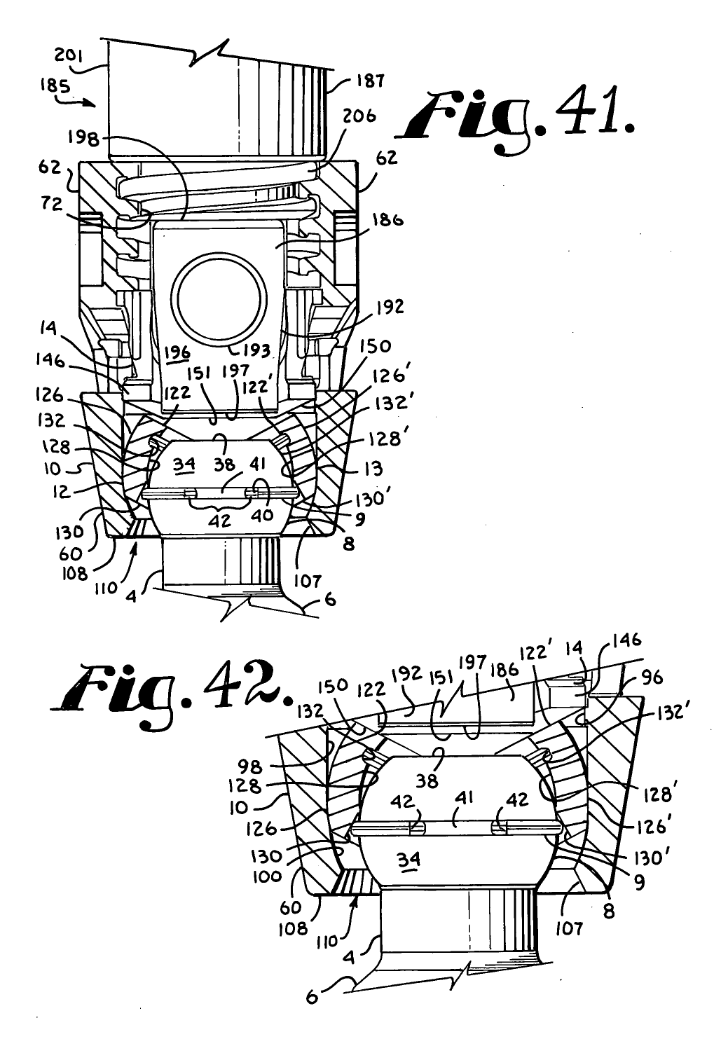

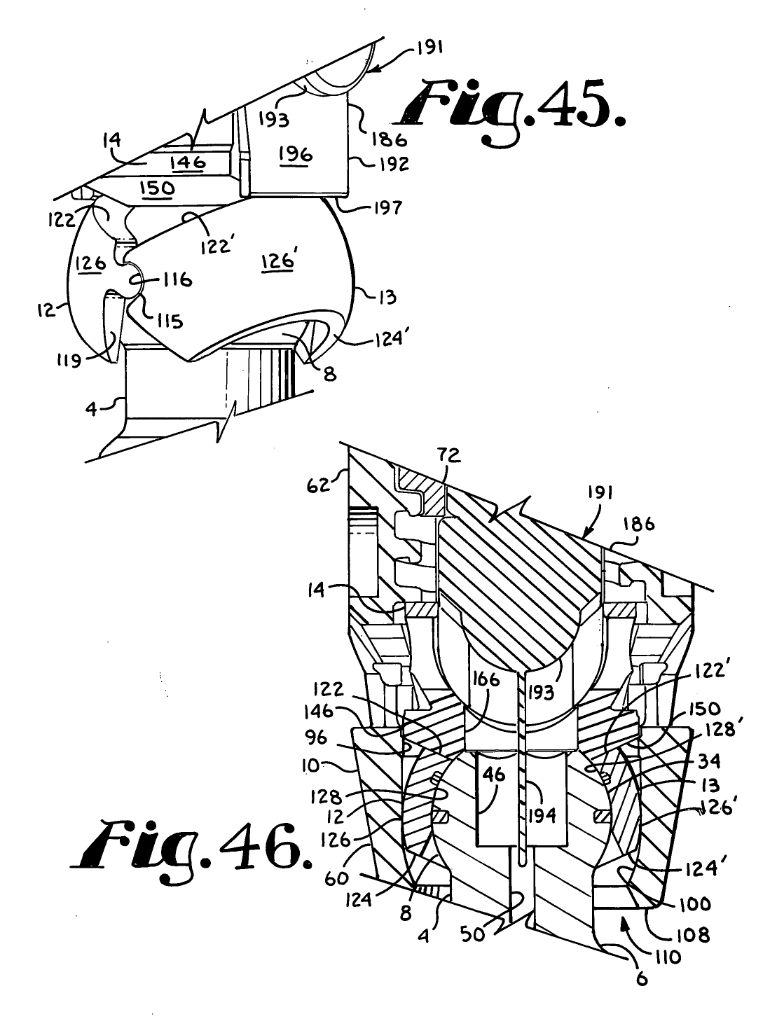

A two-piece tool, generally 185 for assembling the retainer pieces 12, 13 with the shank 4 within the receiver 10 is shown in FIGS. 31-38. The tool 185 includes an inner plunger 186 in sliding cooperation with an outer holder or guide 187. The plunger 186 further includes an upper handle portion 189 integral with a cylindrical shaft 190 sized and shaped for being slidingly received in the tubular holder 187. The shaft 190 terminates at an engagement structure, generally 191, that further includes a pair of opposed extenders or arms 192 projecting downwardly from a cylindrical seating portion 193, the portion 193 perpendicular to the two arms 192. Although only shown in FIG. 46, the tool seating portion 193 preferably includes a centrally located and downwardly extending extension or elongate pin 194 sized for aligning the engagement structure 191 with the shank head 8, the pin 194 aligning with and extending downwardly into the shank cannulation bore 50. The arms 192 include inner 195 and outer 196 cylindrical surfaces terminating at planar bottom surfaces 197. The inner arms surfaces 195 are sized to have a radius slightly larger than an outer radius of the compression insert body portion 136, as shown, for example, in FIG. 45. The illustrated cylindrical seating portion 193 extends through the arm outer surfaces 196. The arm bottom surfaces 197 are opposite and parallel to a top surface 198 of the engagement structure 191, the top surface 198 being adjacent and substantially perpendicular to the shaft 190. The holder 187 is substantially cylindrical and tubular and includes an upper faceted holding surface 200 adjacent to a lower outer cylindrical surface 201 having a diameter smaller than an outer width of the upper surface 200. The holder 187 further includes an inner cylindrical surface 203 sized and shaped for slidingly receiving the plunger shaft 190. The holder 187 has annular top 204 and bottom 205 surfaces. Adjacent the bottom surface 205 is a guide and advancement structure 206 sized and shaped for helical mating cooperation and engagement with the guide and advancement structure 72 of the receiver 10. The plunger 186 and the holder 187 are sized such that when the guide and advancement structure 206 is engaged with the receiver guide and advancement structure 72, the plunger 186 may be pushed down into the receiver 10 a desired distance to press the plunger arm bottom surfaces 197 against the retainer pieces 12 and 13 at respective top surfaces 122 and 122′ thereof to break the weld or other fixing between the pieces 12 and 13 and press down evenly on both of the surfaces 122 and 122′, the interconnected pieces 12 and 13 rotating with respect to one another at the jig-saw-puzzle like connection made up of the grooved portions 116 and adjacent knob portions 115. The knob portions 115 pivot with respect to the grooved portions 116 until the top surfaces 122 and 122′ are disposed in substantially the same plane and the pieces 12 and 13 are disposed about the shank head 8 and the spring ring 9 as will be described in greater detail below.

The retainer pieces 12 and 13 are preferably welded or otherwise fixed to on another at a factory setting in the desired loading geometry as shown, for example in FIGS. 1, 11 and 27. Also, with reference to FIGS. 28-30 the receiver 10, the retainer 12,13 in the temporarily angulated fixed loading geometry and the compression insert 14 may be assembled at a factory setting that includes tooling for holding and alignment of the component pieces as well as compressing or expanding the insert 14 arms, if needed, as well as crimping a portion of the receiver 10 toward the insert 14. In some circumstances, the shank 4 is also assembled with the receiver 10, the retainer 12 and the compression insert 14 at the factory. In other instances, and with reference to FIG. 40, for example, it is desirable to first implant the shank 4, followed by addition of the pre-assembled receiver, retainer and compression insert at the insertion point. In this way, the surgeon may advantageously and more easily implant and manipulate the shanks 4, distract or compress the vertebrae with the shanks and work around the shank upper portions or heads without the cooperating receivers being in the way. In other instances, it is desirable for the surgical staff to pre-assemble a shank of a desired size and/or variety (e.g., surface treatment of roughening the upper portion 8 and/or hydroxyapatite on the shank 6), with the receiver, retainer and compression insert utilizing the tools shown in FIGS. 31-38. Allowing the surgeon to choose the appropriately sized or treated shank 4 advantageously reduces inventory requirements, thus reducing overall cost.

Pre-assembly of the receiver 10, retainer 12 and compression insert 14 is shown in FIGS. 27-30. First, the retainer 12,13 (in the initial attached state) is downloaded in a sideways manner into the receiver 10 through the upper opening 66 with the outer surface 126′ or 126 facing the receiver channel seat 68. The retainer 12 is then lowered between the arms 62 and toward the receiver base 60 as shown in phantom and then in solid lines in FIG. 27, followed by turning or tilting the attached retainer pieces 12,13 to a position within the receiver base 60 inner cavity 61 wherein the retainer bottom surfaces 124 and 124′ are facing the receiver lower opening 110 with the surfaces 126 and 126′ seated upon the inner spherical surface 100 as shown in FIG. 28, the top surfaces 122 and 122′ facing the receiver upper opening 66. With reference to FIG. 28, the compression insert 14 is then downloaded into the receiver 10 through the upper opening 66 with the bottom rim 151 initially facing the receiver arm top surfaces 73 and the insert arms 137 located between the opposed receiver arms 62. The insert 14 is then lowered toward the receiver base 60 until the insert 14 arm upper surfaces 144 are adjacent the run-out area defined by the surfaces 88 of the receiver 10 located below the guide and advancement structure 72. Thereafter, the insert 14 is rotated in a clockwise or counter-clockwise manner about the receiver axis B until the upper arm surfaces 144 are directly below the guide and advancement structure 72 as illustrated in FIG. 29 with the U-shaped channel 141 of the insert 14 aligned with the U-shaped channel 64 of the receiver 10. In some embodiments, the insert arms 137 may need to be compressed slightly during rotation to clear inner surfaces of the receiver arms 62. The outer cylindrical surfaces 143 of the insert 14 are received within the cylindrical surfaces 88 and 90 of the receiver. With particular reference to FIGS. 29 and 30, the receiver thin walls of the crimping area 79 are then pressed inwardly toward the axis B by inserting a tool (not shown) into the receiver apertures 74, the tool pressing the sloped surface walls 77 until the receiver inner wall surfaces 92 engage the insert 14 at each of the grooves 160 and 161 formed into the outer cylindrical surface 143 of each of the insert arms 137. The crimping of the opposed wall surfaces 87 into the groves 160 and 161 keeps the insert 14 U-shaped channel 141 substantially aligned with the receiver U-shaped channel 64, but allows for some upward and downward movement of the insert 14 along the receiver axis B during bottom loading of the shank 4 as shown in FIG. 41, for example. However, such upward and downward movement requires some force, as the four-point frictional engagement between the insert and the receiver advantageously keeps the insert at a desired axial location and is not a floppy or loose sliding engagement. Thus, the crimping of the receiver walls 77 prohibits rotation of the insert 14 about the receiver axis B but allows for limited axial movement of the insert 14 with respect to the receiver 10 along the axis B when some force is exerted to slide the crimped surfaces 87 up or down along the grooves 160 and 161. As illustrated in FIG. 29, the insert 14 arms 137 are fully captured within the receiver 10 by the guide and advancement structure 72 prohibiting movement of the insert 14 up and out through the receiver opening 66 as well as by the retainer 12,13 located in the receiver 10 base 60 below the insert 14. Movement of the retainer 12,13 is also advantageously limited by both the receiver surface 100 and the insert 14. FIGS. 29, 30 and 39 illustrate a preferred arrangement for shipping of the receiver, retainer and insert combination.

At this time, the receiver, insert and retainer combination are ready for shipping to an end user, with both the compression insert 14 and the retainer 12,13 captured within the receiver 10 in a manner that substantially prevents movement or loss of such parts out of the receiver 10. The receiver 10, compression insert 14 and the retainer 12,13 combination may now be assembled with the shank 4 either at the factory, by surgery staff prior to implantation, or directly upon an implanted shank 4 as shown, for example, in FIG. 40, with the shank axis A and the receiver axis B preferably being aligned during assembly as shown in FIGS. 40-50. After assembly with the shank 4, but before insertion of a rod and closure top, the receiver 10 may be placed at an angle with respect to the shank as shown, for example, in FIG. 51.

As illustrated in FIG. 40, the bone screw shank 4 or an entire assembly 1 made up of the assembled shank 4, receiver 10, retainer 12,13 and compression insert 14, is screwed into a bone, such as the vertebra 17, by rotation of the shank 4 using a suitable driving tool (not shown) that operably drives and rotates the shank body 6 by engagement thereof at the internal drive 46. Specifically, the vertebra 17 may be pre-drilled to minimize stressing the bone and have a guide wire (not shown) inserted therein to provide a guide for the placement and angle of the shank 4 with respect to the vertebra. A further tap hole may be made using a tap with the guide wire as a guide. Then, the bone screw shank 4 or the entire assembly 1 is threaded onto the guide wire utilizing the cannulation bore 50 by first threading the wire into the opening at the bottom 28 and then out of the top opening at the drive feature 46. The shank 4 is then driven into the vertebra using the wire as a placement guide. It is foreseen that the shank and other bone screw assembly parts, the rod 21 (also having a central lumen in some embodiments) and the closure top 18 (also with a central bore) can be inserted in a percutaneous or minimally invasive surgical manner, utilizing guide wires. When the shank 4 is driven into the vertebra 17 without the remainder of the assembly 1, the shank 4 may either be driven to a desired final location or may be driven to a location slightly above or proud to provide for ease in assembly with the pre-assembled receiver, compression insert and retainer.

With reference to FIGS. 31 and 39, the tool 185 that includes the plunger 186 already received in the tubular holder 187 is first connected to the pre-assembled receiver, insert and retainer by inserting the engagement structure 191 into the receiver 10 through the top opening 66 as shown in FIG. 31, followed by aligning the holder guide and advancement structure 206 with the receiver guide and advancement structure 72 as shown in FIG. 39. With reference to FIG. 40, the holder 187 is then rotated about the receiver axis B, engaging the guide and advancement structure 206 with the guide and advancement structure 72 of the receiver until the holder cylindrical surface 201 abuts against the receiver arm top surfaces 73. At this time, the engagement structure 191 is located between the receiver arms 62 with the extender arms bottom surfaces 197 shown spaced from the retainer top surfaces 122 and 122′. The plunger 186 is held in this desired upward position by the user during the initial insertion of the shank head 8 into the receiver 10 as shown in FIGS. 40-44, for example.

With further reference to FIGS. 40-44, the pre-assembled receiver, insert and retainer are placed above the shank upper portion 8 (FIG. 40) until the shank upper portion is received within the opening 110. As the shank upper portion 8 is moved into the interior 61 of the receiver base defined by the spherical surface 100, the shank upper portion 8 presses the retainer 12 upwardly into the portion of the receiver cavity 61 defined by the cylindrical surface 98 (FIGS. 41 and 42). At this time, the shank makes contact with the retainer and pushes the retainer pieces upwardly into contact with the insert 14, the retainer top surfaces 122 and 122′ abutting against the insert lower surface 150. Also, with reference to FIG. 46, at this time, alignment between the components of the assembly is maintained by the pin 194 aligning with and entering into the cannulation bore 50 of the shank 4. Initially, as the shank moves upwardly, the spring ring 9 comes into contact with the retainer inner surfaces 130 and 130′ (also shown in FIG. 42). With reference to FIG. 43, continued upward pressure of the shank head 8 moves the retainer inner surfaces 128, 129, 128′ and 129′ downwardly along the spherical shank surfaces 34, compressing the spring 9 into a position flush with the shank head spherical surface 34. With reference to FIGS. 44 and 46, continued upward movement of the shank then results in a flush or concentric contact between the shank head surface 34 located above the spring ring 9 and the inner spherical surfaces 128 and 128′ of the shank pieces 12,13. The upward force required to manipulate the two pieces 12 and 13 into such concentric, flush contact also slightly pivots the knobs 115 with respect to the surfaces of the grooves 116, thus breaking the weld bead or other fixation material holding the pieces 12 and 13 in the initially desired loading position. At this time, the pieces 12 and 13 are still loosely connected with the grooves 116 cooperating with the knobs 115, but the pieces are now movable with respect to each other and are ready for the final plunger deployment step that results in the ring pieces 12 and 13 fully surrounding and retaining the shank head 8 within the receiver cavity 61.

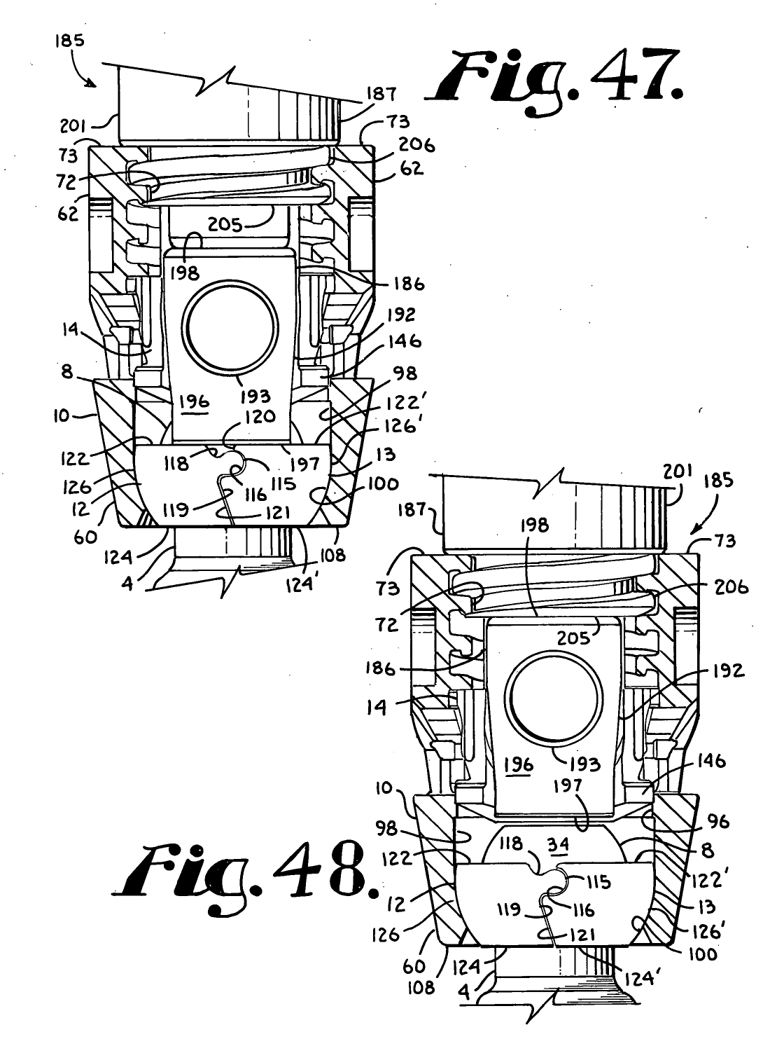

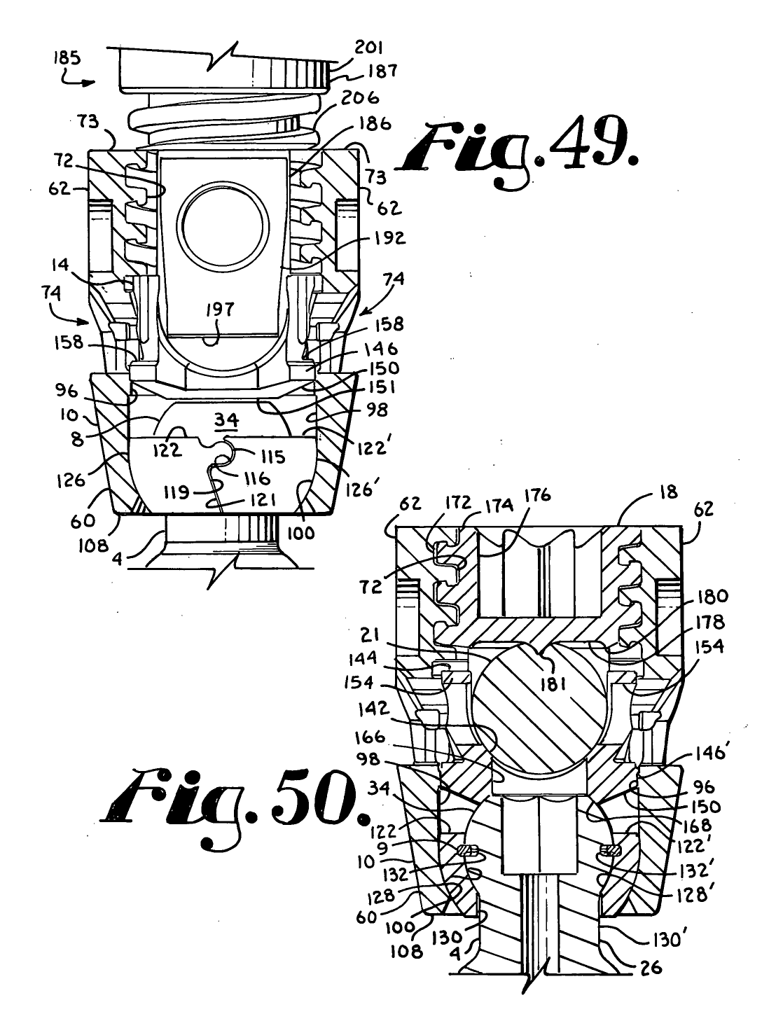

With reference to FIG. 45, it is noted that at this time, the retainer pieces 12 and 13 are aligned with the shank 4 and the insert 14 and thus cannot pivot with respect to the receiver, but the retainer 12,13 is still rotatable about the aligned shank axis A and receiver axis B. Thus, the retainer pieces 12,13 may or may not be in the precise locations shown in FIG. 44 or 45, for example, and thus the nobs 115 and adjacent groove surfaces 116 may or may not be centrally located between the plunger arms 192. Therefore, it is desirable to both push down on the plunger, pushing the upper handle 189 toward the bone screw assembly, and also twist or rotate the tool 185 about the A and B axes, (while holding on to the shank body 6, if such is not already implanted) during a deployment step in which the retainer pieces 12 and 13 are pressed in a downward direction by the plunger arm surfaces 197 moving toward and against the retainer top surfaces 122 and 122′ as well as a spinning movement of the arms about the top surfaces 122 and 122′ to both fully push and sweep the top surfaces 122 and 122′ into a planar arrangement with one another. Preferably, a twist or turn of the tool 185 with respect to the shank 4 of at least one-hundred-eighty degrees is desirable. With reference to FIG. 47, the plunger 186 is shown fully deployed with the retainer top surfaces 122 and 122′ now planar and the retainer outer spherical surfaces 126 and 126′ being fully seated on the spherical surface 100 of the receiver 10. Thereafter, the plunger is retracted as shown in FIG. 48 and the outer tool is rotated about the receiver axis B to unscrew the guide and advancement structure 206 out of the receiver guide and advancement structure 72 as shown in FIG. 49. The entire tool 185 may now be moved out and away from the receiver top opening 66. With reference to FIG. 50, at this time the spring ring 9 returns to a neutral or near neutral state with inner portions thereof located in the groove 39 of the shank head 8 and outer portions thereof located in the grooves 132 and 132′ of the respective interconnected pieces 12 and 13. Also, at this time the cylindrical surfaces 130 and 130′ are located about the neck 26 of the shank 4 located adjacent the head 8.

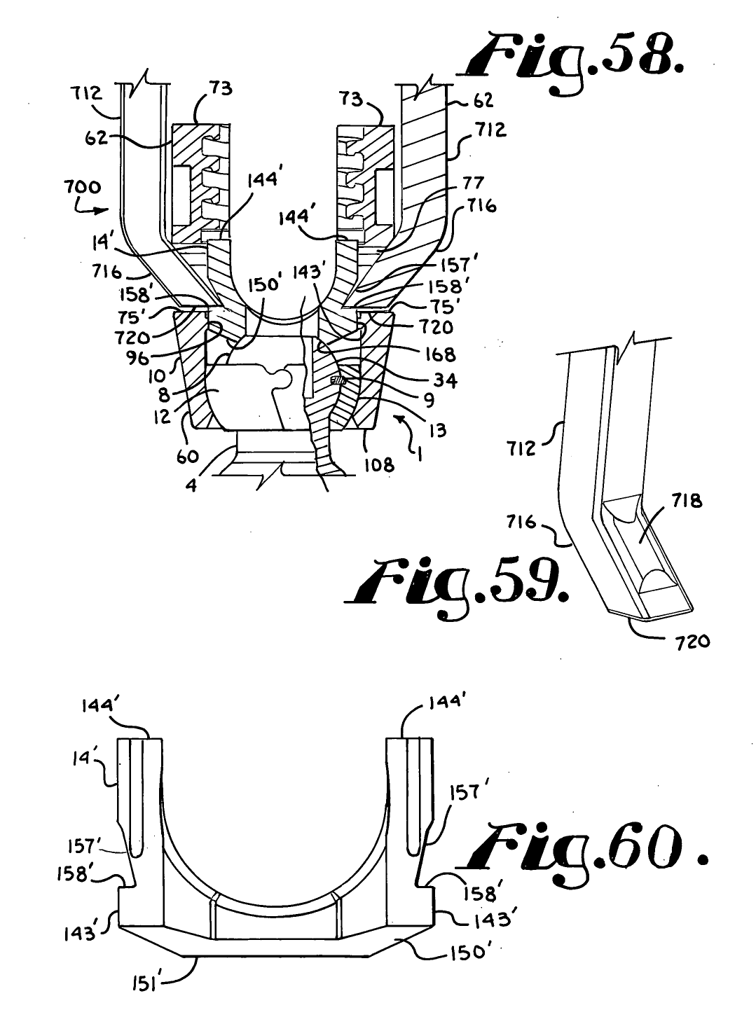

With further reference to FIG. 49, at this time, a tool or tools (for example, tooling with arms the same or similar to the tools 700 shown in FIGS. 58-60) may be used to enter into the delta-shaped apertures 74 of the receiver 10 and press down upon the insert 14 at the surfaces 158 to force the insert cylindrical surface 146 into an interference fit with the cylindrical surface 96 of the receiver 10. Such tools may be used to push down enough to result in an engagement between the insert surface 168 and the shank spherical surface 34 that allows for non-floppy pivoting movement of the shank 4 and attached retainer pieces 12,13 with respect to the insert 14 (and thus with respect to the receiver 10) to a desired angular orientation. Thus, at this time, the receiver 10 may be articulated to a desired angular position with respect to the shank 4, such as that shown in FIG. 51, for example, but prior to insertion of the rod or closure top, that will be held, but not locked, by frictional engagement between the insert 14 (that is now locked against the receiver wall 96) and the shank upper portion 8. In some instances, tools such as the tools 700 may be used to press the insert 14 down into a final frictional locking engagement with the shank head 8, the retainer pieces 12 and 13 expanding outwardly into fixed frictional engagement with the spherical surface 100 of the receiver 10.

With reference to FIG. 50, in the illustrated embodiment, the rod 21 is eventually positioned in an open or percutaneous manner in cooperation with the at least two bone screw assemblies 1. The closure structure 18 is then inserted into and advanced between the arms 62 of each of the receivers 10. The closure structure 18 is rotated, using a tool engaged with the inner drive 176 until a selected pressure is reached at which point the rod 21 engages the U-shaped seating surface 142 of the compression insert 14, pressing the insert surface 168 into locked frictional engagement with the shank spherical surface 34. Specifically, as the closure structure 18 rotates and moves downwardly into the respective receiver 10, the rim 190 engages and penetrates the rod surface 22, the closure structure 18 pressing downwardly against and biasing the rod 21 into compressive engagement with the insert 14 that urges the shank upper portion 8 toward the interconnected retainer 12,13 and into locking engagement therewith, the retainer 12,13 frictionally abutting and expanding outwardly against the spherical surface 100.

Also, in the embodiment illustrated in FIG. 50, the downward force applied by the closure top 18 on the rod 21 presses the insert surfaces 146 into an interference fit arrangement with the receiver cylindrical surface 96. Now, the closure top 18 may be loosened and the rod 21 manipulated with the remainder of the assembly 1 being locked in place as the insert 14 is still pressing downwardly upon the shank surface 34. Also, if desired, the closure top 18 and the rod 21 may be removed as shown in FIG. 52 and replaced with an alternative rod, such as the deformable rod 21′ and alternative closure top 18′ shown in FIGS. 53 and 54 all the while advantageously maintaining a locked angular position of the shank 4 with respect to the receiver 10. It is noted that the closure drive 176′ may advantageously be made smaller than the drive of the closure 18, so that the deformable rod 21′ is not unduly pressed or deformed during assembly since the polyaxial mechanism is already locked.

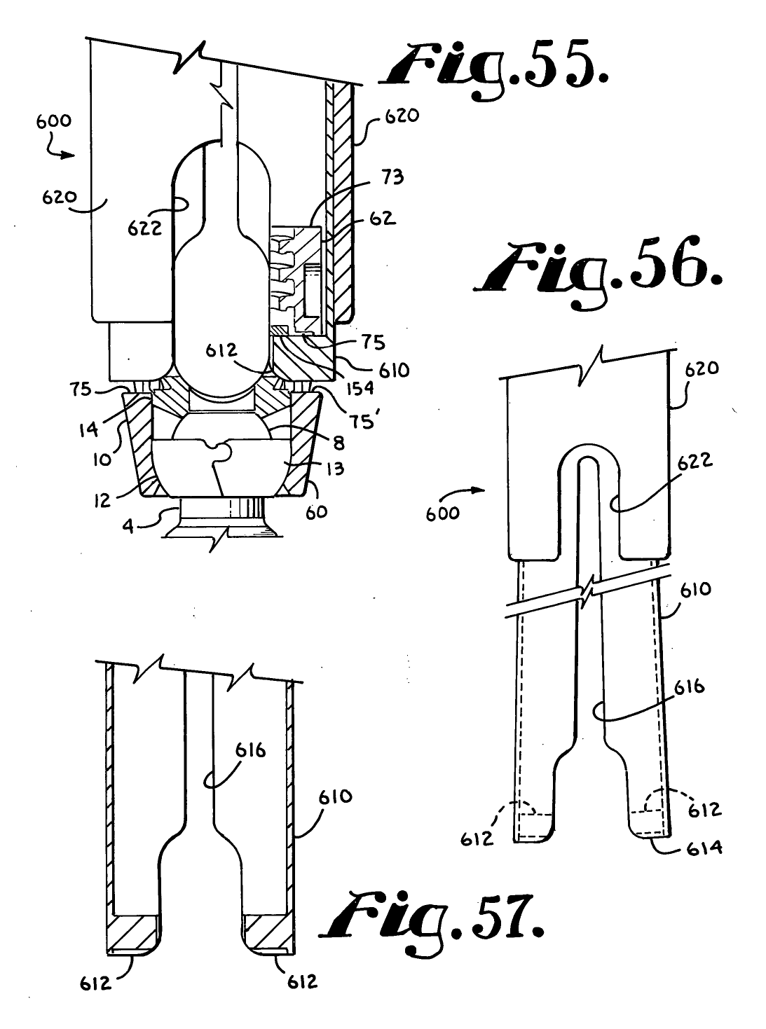

With reference to FIGS. 55-57, a two-piece tool 600 is illustrated for releasing the insert 14 from the receiver 10. The tool 600 includes an inner flexible tube-like structure with opposed inwardly facing prongs 612 located on either side of a through-channel 616. The channel 616 may terminate at a location spaced from the prongs 612 or may extend further upwardly through the tool, resulting in a two-piece tool 610. The tool 600 includes an outer, more rigid tubular member 620 having a smaller through channel 622. The member 620 slidingly fits over the tube 610 after the flexible member 610 prongs 612 are flexed outwardly and then fitted over the receiver 10 and then within through bores of the opposed apertures 74 of the receiver 10 and aligned opposed bores 154 located on the arms of the insert 14. In FIG. 55, the tool 600 is shown during the process of unlocking the insert 14 from the receiver 10 with the outer member 620 surrounding the inner member 610 and holding the prongs 612 within the receiver 10 and insert 14 apertures while the tool 600 is pulled upwardly away from the shank 4. It is foreseen that the tool 600 may further include structure for pressing down upon the receiver 10 while the prongs and tubular member are pulled upwardly, such structure may be located within the tool 600 and press down upon the top surfaces 73 of the receiver arms, for example.

Alternatively, another manipulation tool (not shown) may be used that is inserted into the receiver at the opening 66 and into the insert channel formed by the saddle 141, with prongs or extensions thereof extending outwardly into the insert through bores 154; a piston-like portion of the tool thereafter pushing directly on the shank upper portion 8, thereby pulling the insert 14 away from the receiver surface 96 and thus releasing the polyaxial mechanism. At such time, the shank 4 may be articulated with respect to the receiver 10. If further disassembly of the assembly is desired, such is accomplished in reverse order to the procedure described previously herein for the assembly 1.

With reference to FIGS. 58-60, another manipulation tool, generally 700 is illustrated for independently locking the insert 14 as described earlier, or as shown, for temporarily independently locking a non-locking insert 14′ to the receiver 10. First, with respect to FIG. 58, the non-locking insert 14′ is shown in a bone screw assembly 1′ wherein the insert 14 of the assembly 1 previously described herein is replaced by the non-locking insert 14′. Otherwise, the assembly 1′ is identical to the assembly 1 previously described herein, and therefore includes a shank 4, a spring ring 9, a receiver 10 and interlocking retainer pieces 12 and 13. With respect to FIG. 60, the non-locking insert 14′ is substantially similar in form and function to the previously described locking insert 14. However, the insert 14′ does not include the outer band surfaces 146 found on the arms of the locking insert 14. Rather, outer arms surfaces 143′ of the non-locking insert 14′ extend from top arm surfaces 144′ all the way to lower sloping surfaces 150′ located adjacent a bottom rim 151′. Thus, the arm surfaces 143′ freely slide up and down with respect to the receiver inner surface 96. The non-locking insert 14′ also does not require through bores 154, but does include an opposed pair of notches, each having a sloping surface 157′ and a bottom surface 158′. The insert 14′ otherwise includes all of the other features described herein with respect to the insert 14.

The tool 700 includes a pair of opposed arms 712, each having an engagement extension 716 positioned at an angle with respect to the respective arm 712 such that when the tool is moved downwardly toward the receiver, one or more inner surfaces 718 of the engagement extension 716 slide along the surfaces 77 of the receiver and the surfaces 157′ of the insert 14 to engage the insert 14′, with each surface 720 pressing downwardly on one of the insert surfaces 158′ to lock the polyaxial mechanism of the assembly 1. With reference to FIG. 58, it is noted that when the insert 14 is locked against the receiver 10, the tool bottom surfaces 720 do not bottom out on the receiver surfaces 75′, but remained spaced therefrom. In the illustrated embodiment, the surface 718 is slightly rounded and it is noted that each arm extension 716 may further include surfaces creating an edge with the bottom surface 720 for insertion and gripping of the insert 14′ at the juncture of the surface 157′ and the surface 158′. The tool 700 may include a variety of holding and pushing/pulling mechanisms, such as a pistol grip tool, that may include a ratchet feature, a hinged tool, or, a rotatably threaded device, for example.

It is to be understood that while certain forms of the present invention have been illustrated and described herein, it is not to be limited to the specific forms or arrangement of parts described and shown.

What is claimed and desired to be secured by Letters Patent is as follows:

1. A bone anchor comprising:
a) a shank having an elongate body and an upper portion, the body being configured for fixation to a bone;
b) a receiver having a top portion and a base, the receiver top portion defining a channel for receiving a longitudinal connecting member, the base having an internal seating surface partially defining a cavity, the channel communicating with the cavity, the cavity communicating with an exterior of the base through a receiver opening sized and shaped for upward loading of the shank upper portion through the receiver opening; and
c) a retainer having first and second discrete parts, the parts being attached to one another during loading of the retainer into the receiver, each retainer part having an inner surface and an outer surface, each inner surface configured for fixed engagement with the shank upper portion and each outer surface configured for sliding engagement with the receiver seating surface during pivoting of the shank with respect to the receiver, the retainer parts being detached from one another and each retainer part captured between the shank upper portion and the seating surface during pivoting of the shank with respect to the receiver, the parts cooperating to prevent the shank upper portion from passing down through the receiver opening, the parts being pressed away from one another during fixing of the shank at a desired angular orientation with respect to the receiver.

2. The bone anchor of claim 1 further comprising a compression insert disposed within the receiver and configured to frictionally engage the shank upper portion at a location spaced from the retainer.

3. The bone anchor of claim 2 wherein the insert is in a releasable frictional engagement with a portion of the receiver.

4. The bone anchor of claim 2 wherein the insert has a pair of opposed upwardly extending arms, each arm having a pair of spaced recesses formed in an outer surface of the arm, the recesses sized and shaped for receiving inwardly crimped material from the receiver for prohibiting rotation of the insert with respect to the receiver.

5. The bone anchor of claim 2 wherein the insert is sized and shaped to directly engage and cooperate with lock and release tools, the insert having a first tool receiving sloping surface positioned for alignment with a receiver second tool receiving sloping surface.

6. The bone anchor of claim 1 further comprising a knob on the first retainer part and a groove on the second retainer part, the groove receiving a portion of the knob, and wherein during loading of the retainer into the receiver, the first and second retainer parts being attached to one another at an interface of the knob and the groove.

7. The bone anchor of claim 6 wherein, after the first retainer part is detached from the second retainer part in the receiver, the knob portion remains received within the groove.

8. The bone anchor of claim 1 wherein the first retainer part has a first top surface and the second retainer part has a second top surface, the first and second top surfaces being disposed at an angle with respect to one another when the retainer parts are attached to one another and the first and second top surfaces being substantially in a same plane when the parts are detached from one another.

9. In a bone anchor, the improvement comprising:
a) a shank having a body for fixation to a bone and an integral upper portion having a first spherical surface;
b) a receiver having a top portion and a base, the receiver top portion defining an open channel, the base having a first surface partially defining a cavity, the channel communicating with the cavity;
c) at least one insert disposed within the receiver the insert having a concave surface frictionally mating with the shank first spherical surface;
d) a multi-piece interconnected retainer captured within the cavity, the pieces being movable and detachable while remaining interconnected, the pieces being positionable about at least a portion of the shank, the retainer having a concave surface for fixed engagement with the shank and a convex surface for unlocked slidable articulatable engagement with the receiver; and
e) wherein expansion-only locking engagement occurs between the shank upper portion and the retainer and between the retainer and the receiver.

10. The improvement of claim 9 wherein the insert is in a releasable frictional engagement with a portion of the receiver.

11. The improvement of claim 10 wherein the insert has a pair of opposed upwardly extending arms, each arm having a pair of spaced recesses formed in an outer surface of the arm, the recesses sized and shaped for receiving inwardly crimped material from the receiver for prohibiting rotation of the insert with respect to the receiver.

12. The improvement of claim 11 wherein the insert is sized and shaped to directly engage and cooperate with lock and release tools, the insert having a first tool receiving sloping surface positioned for alignment with a receiver second tool receiving sloping surface.

13. The improvement of claim 9 wherein the multi-piece retainer has first and second pieces and further comprises a knob on the first retainer piece and a groove on the second retainer piece, the groove receiving a portion of the knob, and wherein during loading of the retainer into the receiver, the first and second retainer pieces are attached to one another at an interface of the knob and the groove.

14. The improvement of claim 13 wherein, the first retainer piece is detached from the second retainer piece in the receiver, the knob portion remaining received within the groove.

15. The improvement of claim 13 wherein the first retainer piece has a first top surface and the second retainer piece has a second top surface, the first and second top surfaces being disposed at an angle with respect to one another when the retainer pieces are attached to one another and the first and second top surfaces being substantially in a same plane when the pieces are detached from one another.

16. The improvement of claim 15 further comprising a tool cooperating with the first and second top surfaces, the tool sized and shaped to engage the first and second top surfaces and press the first and second top surfaces downwardly towards the receiver base, the tool pressing the first and second top surfaces into the substantially planar position and detaching the first and second retainer pieces.

17. In a bone anchor, the improvement comprising:
a) a shank having a body for fixation to a bone and an integral upper portion having a first spherical surface;
b) a receiver having a top portion and a base, the receiver top portion defining an open channel, the base having a first surface partially defining a cavity, the channel communicating with the cavity, the receiver also having a first tool receiving sloping surface and through aperture;
c) at least one insert disposed within the receiver, the insert top loaded into the receiver channel and sized and shaped to directly engage and cooperate with lock and release tools, the insert having a second tool receiving sloping surface positioned for alignment with the receiver first tool receiving sloping surface; and
d) a multi-piece interconnected retainer captured within the cavity, the retainer being loadable into the receiver in an attached orientation and movable in a detached orientation to a position surrounding at least a portion of the shank, the retainer being fixed to the shank upper portion and articulatable with respect to the receiver when in an unlocked position, and wherein expansion-only locking engagement occurs between the shank upper portion and the retainer and between the retainer and the receiver.

18. The improvement of claim 17 wherein the interconnected retainer has first and second discrete pieces fixed to one another during loading of the retainer into the receiver.

19. The improvement of claim 18 wherein the first retainer piece has a curved convex projection and the second piece has a curved concave recess, the projection received within the recess and fixed to the second piece during loading of the retainer into the receiver, the projection detached from the second piece and received within the recess during pivoting of the projection within the recess during assembly of the retainer with the shank within the receiver cavity.
